(19) United States
(12) Patent Application Publication
(10) Pub. No.: US 2023/0391817 A1
(43) Pub. Date: Dec. 7, 2023

(54) 5ALPHA-HYDROXY-6BETA-[2-(1-H-IMIDAZOL-4-YL)-ETHYLAMINO]-CHOLESTAN-3BETA-OL ANALOGUES AND PHARMACEUTICAL COMPOSITIONS COMPRISING SAME FOR USE IN THE TREATMENT OF CANCER

(71) Applicant: DENDROGENIX, Liège (BE)

(72) Inventors: Stéphane SILVENTE, Liège (BE); Quentin MARLIER, Liège (BE); Arnaud RIVES, Liège (BE); Nicolas CARON, Liège (BE); Dario MOSCA, Liège (BE); Hélène MICHAUX, Liège (BE)

(73) Assignee: DENDROGENIX, Liège (BE)

(21) Appl. No.: 18/034,317

(22) PCT Filed: Oct. 28, 2021
(86) PCT No.: PCT/EP2021/080054

§ 371 Date: Apr. 27, 2023

(30) Foreign Application Priority Data
Oct. 29, 2020 (BE) 20205764
Dec. 3, 2020 (BE) 20205878

(51) Int. Cl. C07J 43/00; A61K 45/06; A61P 35/02

(52) U.S. Cl. CPC C07J 43/003; A61P 35/02; A61K 45/06

(57) ABSTRACT

The invention relates to a novel compound of general formula (I):

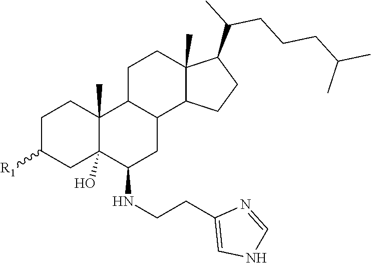

and/or a pharmaceutically acceptable salt of such a compound, to a pharmaceutical composition comprising at least said compound, for its use as a medicament for shrinking a mammalian cancerous tumor.

### FIELD OF THE INVENTION

The invention relates to the field of sterol compounds and more particularly to analogs of the compound 5α-hydroxy-6β-[2-(1H-imidazol-4-yl)ethylamino]cholestan-3β-ol and to pharmaceutical compositions comprising same, for use in the treatment of cancer.

### BACKGROUND OF THE INVENTION

The term “cancer” or “cancerous tumor” encompasses a group of diseases characterized by the uncontrolled multiplication and spread of abnormal cells. If the cancerous cells are not eliminated, the disease will progress more or less rapidly to the death of the affected person.

The management of cancer involves surgery, radiation therapy and chemotherapy, which may be used alone or in combination, simultaneously or sequentially. Chemotherapy uses antineoplastic agents, which are drugs that prevent or inhibit the maturation and proliferation of neoplasms. Antineoplastic agents work by effectively targeting rapidly-dividing cells. Since antineoplastic agents affect cell division, tumours with high growth rates (such as acute myeloid leukemia and aggressive lymphomas, including Hodgkin's disease) are more sensitive to chemotherapy since a greater proportion of the targeted cells are undergoing cell division at any given time. Malignant tumors with slower growth rates, such as indolent lymphomas, tend to respond much more modestly to chemotherapy. However, the development of chemoresistance is an ongoing problem during chemotherapy treatment. For example, conventional treatment of acute myeloid leukemia (AML) involves the combined administration of cytarabine with an anthracycline, such as daunorubicin. The 5-year overall survival rate is 40% in young adults and approximately 10% in elderly patients. Response rates vary considerably with aging, from 40% to 55% in patients over 60 years old and from 24% to 33% in patients over 70 years old. This is even worse for the elderly with unfavorable cytogenetic profiles, and death within 30 days of treatment ranges from 10% to 50% with age and worsening. In addition, restriction of the use of these molecules is also due to side effects, and in particular to the emergence of chronic cardiac toxicity (associated with anthracyclines). The toxic mortality rate associated with intensive chemotherapy is 10% to 20% in patients over 60 years old.

With this benefit-risk profile of the conventional regimen, only 30% of elderly people with a newly diagnosed AML receive antineoplastic chemotherapy.

In the last few decades, there has been only modest improvement in outcomes for younger patients suffering from AML, but none for adults over 60 years old (the majority of patients suffering from AML).

Thus, there is a real need to develop molecules that are useful in the treatment of these cancerous tumors which present problems of chemoresistance and intrinsic toxicity of antineoplastic drugs. The abovementioned data underline the need to find novel approaches which combine both a reduction in the antineoplastic agent dosage regimens for the treatment of chemosensitive tumors with a reduction in the resistance of tumors that are chemoresistant to the antineoplastic agent.

EP3272350B1 discloses the compound 5α-hydroxy-6β-[2-(1H-imidazol-4-yl)ethylamino]cholestan-3β-ol, known as dendrogenin A, and referred to hereinbelow as DX101, which is useful for the treatment of chemoresistant tumors. Dendrogenin A is capable of restoring the sensitivity of chemoresistant tumors to an antineoplastic agent or of enhancing the effects of antineoplastic agents on tumors, which in turn reduces the effective cytotoxic dose of antineoplastic agents against chemosensitive tumors.

The document from De Medina et al. (Biochimie, 2021, 95(3), 482-488, XP028982107, Technical note: Hapten synthesis, antibody production and development of an enzyme-linked immunosorbent assay for detection of the natural steroidal alkaloid dendrogenin A) describes dendrogenin A derivatives for which the alcohol in position 33 is functionalized, for their use as haptens for antibody production.

The document from De Medina et al. (J. Med. Chem., 2009, 52(23), 7765-77, XP9131948, Synthesis of new alkylaminooxysterols with potent cell differentiating activities: identification of leads for the treatment of cancer and neurodegenerative diseases) describes dendrogenin A derivatives for which the alcohol in position 3β is optionally functionalized with a methoxide or propoxide radical, for the treatment of cancer.

The object of the present invention is to provide novel compounds and analogs of the compound dendrogenin A, which are useful for treating cancerous tumors, notably chemosensitive and/or chemoresistant tumors.

Surprisingly, the inventors have discovered that specific analogs of the compound dendrogenin A (also named DX101) show pharmacological activity comparable to that of dendrogenin A.

### SUMMARY OF THE INVENTION

A first subject of the invention is a compound of formula (I):

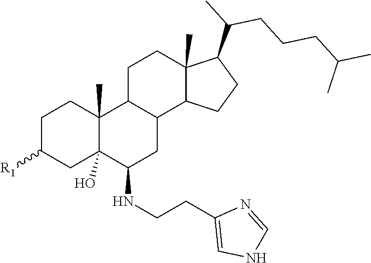

or a pharmaceutically acceptable salt of such a compound,
in which:
R₁ is chosen from F, N₃, OCₙH₂ₙ₊₁, NR₂R₃, SR₂, SO₂R₂, with n≤8,
R₂ and R₃ are independently chosen from: H, a saturated or unsaturated C1 to C8 alkyl group, optionally containing one or more substituents chosen from allyl, carbonyl, arene and heterocyclic groups,
for use as a medicament, and more particularly as a medicament for shrinking a mammalian cancerous tumor.

A second subject of the invention is a pharmaceutical composition comprising, in a pharmaceutically acceptable vehicle, at least one compound of formula (I) for use in shrinking a mammalian cancerous tumor.

In this description, unless otherwise specified, it is understood that when a range is given, it includes the upper and lower limits of said range.

In the present invention, throughout this description and the appended claims, the following terms, unless otherwise indicated, are to be understood as having the following meanings:

The term “solvate” is used herein to describe a molecular complex comprising a compound of the invention and containing stoichiometric or substoichiometric amounts of one or more molecules of a pharmaceutically acceptable solvent such as ethanol. The term “hydrate” refers to when said solvent is water.

The term “human” refers to a subject of either sex and at any stage of development (i.e. newborn, infant, juvenile, adolescent, adult).

The term “patient” refers to a warm-blooded animal, more preferably a human, who is awaiting or receiving medical care and/or who will be the subject of a medical procedure.

The term “pharmaceutically acceptable” means that the ingredients of a pharmaceutically acceptable product are mutually compatible and are not harmful to the patient receiving said product.

The term “pharmaceutical vehicle” as used herein means an inert support or medium used as a solvent or diluent in which the pharmaceutically active agent is formulated and/or administered. Nonlimiting examples of pharmaceutical vehicles include creams, gels, lotions, solutions and liposomes.

The term “administration” means to deliver, the active agent or active ingredient (for example the compound of formula (I)), in a pharmaceutically acceptable composition, to the patient in which a condition, symptom and/or disease is to be treated.

The terms “treat” and “treatment” as used herein include attenuating, alleviating, stopping or caring for a condition, symptom and/or disease.

The term “analog” as used herein means a compound having a chemical structure similar to another reference compound, but differing therefrom in a certain component. It may differ in one or more atoms, functional groups or substructures, which are replaced with other atoms, functional groups or substructures. The analogs may have different physical, chemical, biochemical or pharmacological properties. In the present invention, the analogous compounds are in reference to the compound dendrogenin A. These analogs have the same or similar pharmacological properties relative to the reference compound.

The term “chemoresistant cancer” means a cancer in a patient where the proliferation of the cancer cells cannot be prevented or inhibited with an antineoplastic agent or a combination of antineoplastic agents normally used for treating said cancer, at a dose that is acceptable to the patient. The tumors may be inherently resistant prior to chemotherapy, or resistance may be acquired during treatment by tumors that are initially sensitive to chemotherapy.

The term “chemosensitive cancer” means a cancer in a patient that responds to the effects of an antineoplastic agent, i.e., the proliferation of cancer cells can be prevented by means of said antineoplastic agent at a dose that is acceptable to the patient.

The compound of formula (I) belongs to the steroid group. The numbering of the carbon atoms of the compound of formula (I) thus follows the nomenclature defined by IUPAC in Pure & Appl. Chem., Vol. 61, No. 10, pages.1783-1822, 1989. The numbering of the carbon atoms of a compound belonging to the steroid group according to IUPAC is illustrated below:

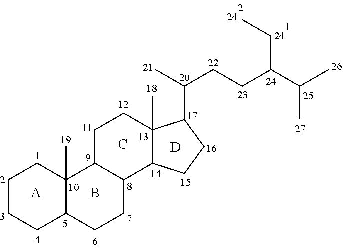

In the present invention, the following abbreviations have the meanings given below:
AML: acute myeloid leukemia;
dendrogenin A: 5α-hydroxy-6β-[2-(1H-imidazol-4-yl)ethylamino]cholestan-3β-ol;
MCF-7: Michigan Cancer Foundation-7;
DMEM: Dulbecco's Modified Eagle Medium;
FCS: fetal calf serum;
ChEH: Cholesterol Epoxide Hydrolase;
Neuro2a: murine neuroblastoma;
CTL: control;
MTT: 3-(4,5-dimethylthiazol-2-yl)-2,5-diphenyltetrazolium bromide;
PBS: phosphate-buffered saline;
DMSO: dimethyl sulfoxide;
OD: optical density or absorbance;
CT: cholestane-3β,5α,6β-triol;
OCDO: 6-oxocholestane-3β,5α-diol;
5,6α-EC: 5,6α-epoxycholesterol;
Tam: tamoxifen;
TLC: thin layer chromatography;
P.O.: per os;
LC/MS: liquid chromatography/mass spectrometry

### BRIEF DESCRIPTION OF THE DRAWINGS

The invention will be better understood and other aims, details, features and advantages thereof will appear more clearly from the following description of several particular embodiments of the invention, given merely for illustration and without limitation, with reference to the attached drawings.

FIG. 1 represents the results of a cytotoxicity study of 3β3-fluoro-5α-hydroxy-6β-[2-(1H-imidazol-4-yl)ethylamino]cholestane (DX111) on Neuro2a cells via a trypan blue assay.

FIG. 2 shows the results of an MTT cell viability assay performed on MCF-7 breast tumor cells in the presence of the compound 3β-fluoro-5α-hydroxy-6β-[2-(1H-imidazol-4-yl)ethylamino]cholestane.

FIG. 3 shows the results of Cholesterol Epoxide Hydrolase (ChEH) activity in MCF-7 cells in the presence of the compound 3β-fluoro-5α-hydroxy-6β-[2-(1H-imidazol-4-yl)ethylamino]cholestane.

FIG. 4 shows the pharmacokinetic profile of the compound 3β-methoxy-5α-hydroxy-6β-[2-(1H-imidazol-4-yl)ethylamino]cholestane (DX103) in comparison with the compound dendrogenin A (DX101).

FIG. 5 shows the pharmacokinetic profile of the compound 3β-ethoxy-5α-hydroxy-6β-[2-(1H-imidazol-4-yl)ethylamino]cholestane (DX105) in comparison with the compound dendrogenin A (DX101).

FIG. 6 shows the pharmacokinetic profile of the compound 3β-fluoro-5α-hydroxy-6β-[2-(1H-imidazol-4-yl)ethylamino]cholestane (DX111) in comparison with the compound dendrogenin A (DX101).

FIGS. 7A and 7B illustrate the evolution of tumor growth and the survival rate in mice comparing treatment with DX111 and DX101.

FIG. 8 shows the pharmacokinetic profile of the compound 3β-azido-5α-hydroxy-6β-[2-(1H-imidazol-4-yl)ethylamino]cholestane (DX123) in comparison with the compound dendrogenin A (DX101).

### DETAILED DESCRIPTION OF THE PREFERRED EMBODIMENTS

A first subject of the invention is a compound of formula (I):

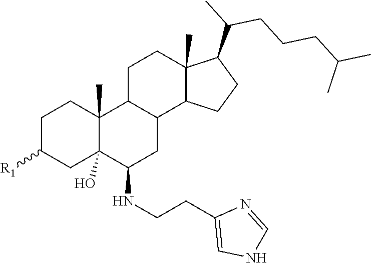

or a pharmaceutically acceptable salt of such a compound, in which:
R₁ is chosen from F, N₃, OCₙH₂ₙ₊₁, NR₂R₃, SR₂, SO₂R₂, with n≤8,
R₂ and R₃ are independently chosen from: H, a saturated or unsaturated C1 to C8 alkyl group, optionally containing one or more substituents chosen from allyl, carbonyl, arene and heterocyclic groups,
for use as a medicament.

According to one embodiment, the invention relates to a compound of formula (I):

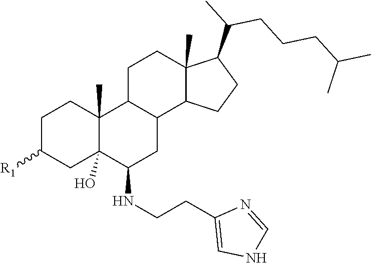

or a pharmaceutically acceptable salt of such a compound, in which:
R₁ is chosen from F, N₃, OCₙH₂ₙ₊₁, NR₂R₃, SR₂, SO₂R₂, with n≤8,
R₂ and R₃ are independently chosen from: H, a saturated or unsaturated C1 to C8 alkyl group, optionally containing one or more substituents chosen from allyl, carbonyl, arene and heterocyclic groups,
for use as a medicament for shrinking a mammalian cancerous tumor.

In the present invention:
the term “carbonyl group” refers to all functional groups containing the oxo group (an oxygen atom doubly bonded (═O) to a carbon atom) and may be chosen from aldehydes, ketones, carboxylic acid, esters, amides and/or anhydride;
the term “allyl” refers to an alkene functional group of the semi-developed formula H₂C═CH—CH₂—;
the term “sulfonyl” refers to a chemical compound in which the sulfur atom is combined with two double bonded oxygen atoms (═O) and with its radical;
the term “arene” refers to all monocyclic and polycyclic aromatic hydrocarbons;
the term “heterocyclic” refers to monocyclic and polycyclic aromatic compounds including as ring members one or more heteroatoms from among O, S and/or N.
   In the definition of the compound of formula (I) according to the invention, the carbon 3 radical may be in the α or β position, the β position being a preferred embodiment.

According to one embodiment, the compound of formula (I) is an O-amino analog in which the radical R₁═NR₂R₃ with R₂ being H or COCₙH₂ₙ₊₁ and R₃═H.

In this embodiment, the compound of formula (I) is more particularly 5α-hydroxy-6β-[2-(1H-imidazol-4-yl)ethylamino]-3β-acetamide (named DX127).

In this embodiment, the compound of formula (I) is more particularly 5α-hydroxy-6β-[2-(1H-imidazol-4-yl)ethylamino]-3β-amine (named DX125).

In this embodiment, the compound of formula (I) is more particularly 5α-hydroxy-6β-[2-(1H-imidazol-4-yl)ethylamino]-3β-azide (named DX123).

According to yet another embodiment, the compound of formula (I) is 3β-fluoro-5α-hydroxy-6β-[2-(1H-imidazol-4-yl)ethylamino]cholestane (named DX111).

According to yet another embodiment, the compound of formula (I) is an O-alkyl analog and has a radical R₁═OCₙH₂ₙ₊₁ with n≤8, and is chosen from:
3β-methoxy-5α-hydroxy-6β-[2-(1H-imidazol-4-yl)ethylamino]cholestane (named DX103)
3β-ethoxy-5α-hydroxy-6β-[2-(1H-imidazol-4-yl)ethylamino]cholestane (named DX105)
3β-octanoxy-5α-hydroxy-6β-[2-(1H-imidazol-4-yl)ethylamino]cholestane (named DX115)

Even more preferentially, the compound of formula (I) is an O-alkyl analog such as 3β-methoxy-5α-hydroxy-6β-[2-(1H-imidazol-4-yl)ethylamino]cholestane (DX103) and 3β-ethoxy-5α-hydroxy-6β-[2-(1H-imidazol-4-yl)ethylamino]cholestane (DX105).
According to a further embodiment, the compound of formula (I) is a sulfur analog and has a radical R₁═SO₂R₂ with R₂ being H or OCₙH₂ₙ₊₁, with n≤8.

In this embodiment, the compound of formula (I) is preferentially 3β-methylsulfonyl-5α-hydroxy-6β-[2-(1H-imidazol-4-yl)ethylamino]cholestane (named DX129).

According to one embodiment, the compound of formula (I) is intended for use in the treatment of cancer of the breast, prostate, colorectal, lung, bladder, skin, uterus, cervix, mouth, brain, stomach, liver, throat, larynx, esophagus, bone, ovary, pancreas, kidney, retina, sinus, nasal cavity, testicle, thyroid, vulva, for the treatment of lymphoma, non-Hodgkin's lymphoma, Hodgkin's lymphoma, leukemia, acute myeloid leukemia or acute lymphocytic leukemia, multiple myeloma, Merkel cell carcinoma or mesothelioma.

According to one embodiment, the cancer is an acinar adenocarcinoma, acinar carcinoma, acro-lentiginous melanoma, actinic keratosis, adenocarcinoma, adenoid cystic carcinoma, adenosquamous carcinoma, adnexal carcinoma, adrenocortical resting tumor, adrenocortical carcinoma, aldosterone-secreting carcinoma, alveolar soft tissue sarcoma, ameloblastic carcinoma of the thyroid, angiosarcoma, apocrine carcinoma, Askin's tumor, astrocytoma, basal cell carcinoma, basaloid carcinoma, basosquamous carcinoma, biliary tract cancer, bone marrow cancer, botryoid sarcoma, bronchioalveolar carcinoma, bronchogenic adenocarcinoma, bronchogenic carcinoma, ex pleomorphic adenoma, chloroma, cholangiocellular carcinoma, chondrosarcoma, choriocarcinoma, choroid plexus carcinoma, clear cell adenocarcinoma, colon cancer, comedocarcinoma, cortisol-producing carcinoma, columnar cell carcinoma, differentiated liposarcoma, ductal adenocarcinoma of the prostate, ductal carcinoma, in situ ductal carcinoma, duodenal cancer, eccrine carcinoma, embryonal carcinoma, endometrial carcinoma, endometrial stromal carcinoma, epithelioid sarcoma, Ewing's sarcoma, exophytic carcinoma, fibroblastic sarcoma, fibrocarcinoma, fibrolamellar carcinoma, fibrosarcoma, follicular thyroid carcinoma, gallbladder cancer, gastric adenocarcinoma, giant cell carcinoma, giant cell sarcoma, giant cell bone tumor, glioma, glioblastoma multiforme, granulosa cell carcinoma, head and neck cancer, hemangioma, hemangiosarcoma, hepatoblastoma, hepatocellular carcinoma, Hurthle cell carcinoma, ileal cancer, lobular infiltrating carcinoma, inflammatory breast carcinoma, intraductal carcinoma, intraepidermal carcinoma, jejunum cancer, Kaposi's sarcoma, Krukenberg tumor, Kulchitsky cell carcinoma, Kupffer cell sarcoma, large cell carcinoma, laryngeal cancer, lentigo maligna melanoma, liposarcoma, lobular carcinoma, in situ lobular carcinoma, lymphoepithelioma, lymphosarcoma, malignant melanoma, medullary carcinoma, medullary thyroid carcinoma, medulloblastoma, meningeal carcinoma, micropapillary carcinoma, mixed cell sarcoma, mucinous carcinoma, mucoepidermoid carcinoma, mucosal melanoma, myxoid liposarcoma, myxosarcoma, nasopharyngeal carcinoma, nephroblastoma, neuroblastoma, nodular melanoma, non-clear cell kidney cancer, non-small cell lung cancer, oat cell carcinoma, ocular melanoma, oral cancer, osteoid carcinoma, osteosarcoma, ovarian cancer, Paget's carcinoma, pancreatoblastoma, papillary adenocarcinoma, papillary carcinoma, papillary thyroid carcinoma, pelvic cancer, periampullary carcinoma, phyllodes tumor, pituitary cancer, pleomorphic liposarcoma, pleuropulmonary blastoma, primary intraosseous carcinoma, rectal cancer, renal cell carcinoma, retinoblastoma, rhabdomyosarcoma, round cell liposarcoma, scar cancer, schistosomal bladder cancer, schneiderial carcinoma, sebaceous carcinoma, ring cell carcinoma, skin cancer, small cell lung cancer, small cell osteosarcoma, soft tissue sarcoma, spindle cell sarcoma, squamous cell carcinoma, stomach cancer, superficial spreading melanoma, synovial sarcoma, telangiectatic sarcoma, terminal duct carcinoma, testicular cancer, thyroid cancer, transitional cell carcinoma, tubular carcinoma, tumorigenic melanoma, undifferentiated carcinoma, adenocarcinoma of the urethra, bladder cancer, uterine cancer, carcinoma of the uterus, melanoma of the uterus, vaginal cancer, verrucous carcinoma, villous carcinoma, well-differentiated liposarcoma, Wilms' tumor or germ cell tumors.

In a preferred embodiment, the compound of formula (I) is intended for use in the treatment of mammalian breast cancer.

According to one embodiment, the compound is intended for use in the treatment of a chemosensitive cancer.

According to a particularly preferred embodiment, the compound of formula (I) is intended for use in the treatment of a chemoresistant cancer.

According to one embodiment, the chemoresistant cancer is a hematological or blood cancer, such as leukemia, in particular acute myeloid leukemia or acute lymphocytic leukemia, lymphoma, in particular non-Hodgkin's lymphoma and multiple myeloma.

According to one embodiment, the cancer is chemoresistant to daunorubicin, cytarabine, fluorouracil, cisplatin, all-trans-retinoic acid, arsenic trioxide, bortezomib, or any combination thereof.

All references to the compounds of formula (I) include references to the salts, multi-component complexes and liquid crystals thereof. All references to the compounds of formula (I) also include references to the polymorphs and the usual crystals thereof.

The compound according to the invention may be in the form of pharmaceutically acceptable salts. A pharmaceutically acceptable salt of the compound of formula (I) comprises the acid addition thereof.

Suitable acid salts are formed from acids which form nontoxic salts, for example chosen from: acetate, adipate, benzoate, bicarbonate, carbonate, bisulfate, sulfate, borate, camsylate, citrate, cyclamate, edisylate, esylate, formate, furamate, gluceptate, gluconate, glucuronate, hexafluorophosphate, hibenzate, chloride hydrochloride, hydrobromide, bromide, hydriodide, iodide, isethionate, lactate, malate, maleate, malonate mesylate, methyl sulfate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate salts, tosylate, trifluoroacetate, and xinofoate. Preferably, the pharmaceutically acceptable salt of the compound of formula (I) is formed from lactate.

The pharmaceutically acceptable salts of the compounds of formula (I) may be prepared via one or more of the following three methods:
(i) reacting the compound of formula (I) with the desired acid;
(ii) removing an acid-labile or base-labile protecting group from a suitable precursor of the compound of formula (I) or ring opening of a suitable cyclic precursor, for example a lactone or lactam, using the desired acid or base; or
(iii) by converting one salt of the compound of formula (I) into another by reaction with a suitable acid or base or by means of a suitable ion exchange column.

These three reactions are usually performed in solution. The obtained salt may precipitate and be collected by filtration or may be recovered by evaporating off the solvent. The degree of ionization of the salt obtained may vary from fully ionized to almost non-ionized.

A second subject of the invention is a pharmaceutical composition comprising, in a pharmaceutically acceptable vehicle, at least one compound according to the invention, as described above, for use in shrinking a mammalian cancerous tumor.

According to one embodiment, the pharmaceutical composition also comprises at least one other therapeutic agent.

According to a preferred embodiment, this other therapeutic agent is an antineoplastic agent.

According to one embodiment, the antineoplastic agent is a DNA-damaging agent such as camptothecin, irinotecan, topotecan, amsacrine, etoposide, etoposide phosphate, teniposide, cisplatin, carboplatin, oxaliplatin, cyclophosphamide, chlorambucil, chlormethine, busulfan, treosulfan or thiotepa, an antitumor antibiotic such as daunorubicin, doxorubicin, epirubicin, idarubicin mitoxantrone, valrubicin, actinomycin D, mitomycin, bleomycin or plicamycin, an antimetabolite such as 5-fluorouracil, cytarabine, fludarabine or methotrexate, an antimitotic such as paclitaxel docetaxel, vinblastine, vincristine, vindesine or vinorelbine, or various antineoplastic agents such as bortezomib, all-trans-retinoic acid, arsenic trioxide, or a combination thereof.

According to one embodiment, the pharmaceutical composition is used in the treatment of cancer in a patient suffering from a tumor that is chemoresistant to said antineoplastic agent when not administered in combination with a compound according to the invention.

According to one embodiment, the pharmaceutical composition is used in the treatment of cancer in a patient suffering from a tumor that is chemosensitive to said antineoplastic agent, and the dose of the antineoplastic agent administered to said patient in combination with a compound according to the invention or a pharmaceutically acceptable salt thereof is less than the dose of the antineoplastic agent when not administered in combination with a compound according to the invention. In particular, the dose of the antineoplastic agent administered to said patient in combination with a compound according to the invention or a pharmaceutically acceptable salt thereof is lower than the dose of the antineoplastic agent administered alone, without any other active principle.

The pharmaceutical composition according to the invention may also further comprise other therapeutically active compounds commonly used in the treatment of the above-stated pathology.

According to one embodiment, the pharmaceutical composition of the invention may be administered via any route, notably including: intradermal, intramuscular, intraperitoneal, intravenous or subcutaneous, pulmonary, transmucosal (oral, intranasal, intravaginal, rectal), nasal spray inhalation, using a tablet, capsule, solution, powder, gel or particle formulation; and contained in a syringe, implanted device, osmotic pump, cartridge or micropump; or any other means as appreciated by the skilled artisan, well known in the art. Site-specific administration may be performed, for example, intratumoral, intra-articular, intrabronchial, intra-abdominal, intracapsular, intracartilaginous, intracavitary, intracerebellar, intracerebroventricular, intracolic, intracervical, intragastric, intrahepatic, intracardiac, intraosteal, intrapelvic, intrapericardial intraperitoneal, intrapleural, intraprostatic, intrapulmonary, intrarectal, intrarenal, intraretinal, intrasynovial, intrathoracic, intrauterine, intravascular, intravesical, intralesional, vaginal, rectal, buccal, sublingual, intranasal, or transdermal in a suitable dosage comprising the usual nontoxic and pharmaceutically acceptable vehicles. Preferably, the pharmaceutical composition is in a form that is suitable for intravenous, subcutaneous, intraperitoneal or oral administration, the oral route being particularly preferred.

In addition to warm-blooded animals such as mice, rats, dogs, cats, sheep, horses, cows and monkeys, the compound of the invention is also effective on humans.

According to one embodiment, the pharmaceutical compositions for administering the compounds of this invention may be presented in unit dose form and may be prepared via any of the methods well known in the prior art. All the methods include the step of placing the active principle in combination with the support which constitutes one or more accessory ingredients. In general, the pharmaceutical compositions are prepared by placing the active ingredient in combination with a liquid support or a finely divided solid support or both and then, if necessary, shaping the product into the desired formulation. In the pharmaceutical composition, the active object compound is included in an amount that is sufficient to produce the desired effect on the disease process or condition. The pharmaceutical compositions containing the active principle may be in a form that is suitable for oral use, for example in the form of tablets, lozenges, aqueous or oily suspensions, dispersible powders or granules, emulsions, capsules, syrups, elixirs, solutions, oral patches, oral gel, chewing gum, chewable tablets, effervescent powder and effervescent tablets. The pharmaceutical compositions containing the active principle may be in the form of an aqueous or oily suspension.

According to one embodiment, the aqueous suspensions contain the active materials in admixture with excipients that are suitable for the manufacture of aqueous suspensions. These excipients are suspending agents, for example sodium carboxymethylcellulose, methylcellulose, hydroxypropylmethylcellulose, sodium alginate, polyvinylpyrrolidone, tragacanth gum and acacia gum; the dispersing or wetting agents may be a natural phosphatide, for example lecithin, or products of condensation of an alkylene oxide with fatty acids, for example polyoxyethylene stearate, or products of condensation of ethylene oxide with long-chain aliphatic alcohols, for example heptadecaethyleneoxyketanol or products of condensation of ethylene oxide with partial esters derived from fatty acids and a hexitol, such as polyoxyethylene sorbitol monooleate, or products of condensation of ethylene oxide with partial esters derived from fatty acids and hexitol anhydrides, for example polyethylene sorbitol monooleate. The aqueous suspensions may also contain one or more preserving agents, for example ethyl or n-propyl p-hydroxybenzoate, one or more colorants, one or more flavorings, and one or more sweeteners, such as sucrose or saccharin.

According to one embodiment, the oily suspensions may be formulated by suspending the active principle in a plant oil, such as groundnut, olive, sesame or coconut oil, or in a mineral oil such as liquid paraffin. The oily suspensions may contain a thickener, for example beeswax, hard paraffin or cetyl alcohol. Sweeteners such as those mentioned above and flavoring agents may be added to obtain a pleasant-tasting oral preparation. These compositions can be preserved by adding an antioxidant such as ascorbic acid. Dispersible powders and granules that are suitable for the preparation of an aqueous suspension by addition of water provide the active ingredient in admixture with a dispersing or wetting agent, a suspending agent and one or more preserving agents.

Syrups and elixirs may be formulated with sweeteners, for example glycerol, propylene glycol, sorbitol or sucrose. These formulations may also contain an emollient, a preserving agent, flavorings and colorants.

The pharmaceutical compositions may be in the form of an aqueous or oleaginous suspension that can be injected in a sterile manner. This suspension may be formulated according to the known art using the suitable dispersing or wetting agents and suspending agents mentioned above. The injectable sterile preparation may also be an injectable sterile solution or suspension in a parenterally acceptable nontoxic diluent or solvent, for example a solution in 1,3-butanediol.

Acceptable vehicles and solvents that may be used include; water, Ringer's fluid and isotonic sodium chloride solution. In addition, sterile fixed oils are conventionally used as solvent or suspension medium. For this purpose, any fixed oil may be used, including synthetic mono- or diglycerides. In addition, fatty acids such as oleic acid are used in the preparation of injectable products.

The pharmaceutical compositions of the present invention may also be administered in the form of suppositories for rectal administration of the medicament. These compositions can be prepared by mixing the medicament with a suitable non-irritant excipient which is solid at ordinary temperature but liquid at the rectal temperature and will therefore melt in the rectum to release the medicament. Such materials include cocoa butter and polyethylene glycols.

In addition, the pharmaceutical compositions can be administered ocularly by means of solutions or ointments. Furthermore, transdermal administration of the compounds under consideration can be achieved by means of iontophoretic patches and the like. For topical use, creams, ointments, jellies, solutions or suspensions are used.

In the treatment of a mammal or patient suffering from or at risk of developing a cancer, an appropriate dosage of the pharmaceutical composition of this invention may generally be from about 0.1 to 50 000 micrograms (μg) per kg of patient body weight per day, which may be administered in single or multiple doses. The dosage level will preferably be from about 1000 to about 40 000 μg/kg per day, depending on many factors such as the severity of the cancer to be treated, the age and relative health of the subject, the route and form of administration. For oral administration, this composition may be provided in the form of tablets containing 1000 to 10 0000 micrograms of each of the active principles, in particular 1000, 5000, 10 000, 15 000, 20 000, 25 000, 50 000, 75 000 or 100 000 micrograms of each active principle. This composition can be administered in a schedule of 1 to 4 times per day, for example once or twice per day. The dosage regimen can be adjusted to provide an optimum therapeutic response.

The invention also discloses a process for manufacturing the compound of formula (I).

According to one embodiment, the C3 fluorination process comprises a step of fluorination of dendrogenin A, performed with a fluorinating reagent, for example diethylaminosulfur trifluoride (DAST) or tetrafluoroborate. The fluorination reaction with DAST is described in the literature: Tetrahedron letters 1979, 20, 1823-1826, “A new method for fluorination of sterols” (https://doi.org/i0.1016/S0040-4039(01)86228-6). The fluorination reaction with tetrafluoroborate is described in the literature: Org. Lett., Vol. 11, No. 21, 2009, 5050-5053, “Aminodifluorosulfinium Tetrafluoroborate Salts as Stable and Crystalline Deoxofluorinating Reagents”.

According to one embodiment, the process for the synthesis of 3β-fluoro-5α-hydroxy-6β-[2-(1H-imidazol-4-yl)ethylamino]cholestane dilactate comprises the following steps:
dissolving the compound 3β-fluoro-5α-hydroxy-6β-[2-(1H-imidazol-4-yl)ethylamino]-cholestane in anhydrous ethanol and then adding lactic acid thereto;
stirring the mixture at room temperature for 3 h;
evaporating off the organic solvent.
   The white powder obtained is the compound 3β-fluoro-5α-hydroxy-6β-[2-(1H-imidazol-4-yl)ethylamino]cholestane dilactate.

According to one embodiment of the process, the ambient temperature is between 15 and 40° C., for example 25 or 37, preferentially 20° C.

### EXAMPLES

Various experiments were performed to evaluate the characteristics of the compounds of formula (I).

The preferred compounds according to the invention corresponding to the general formula I, the synthesis and activity of which are described below, are as follows:

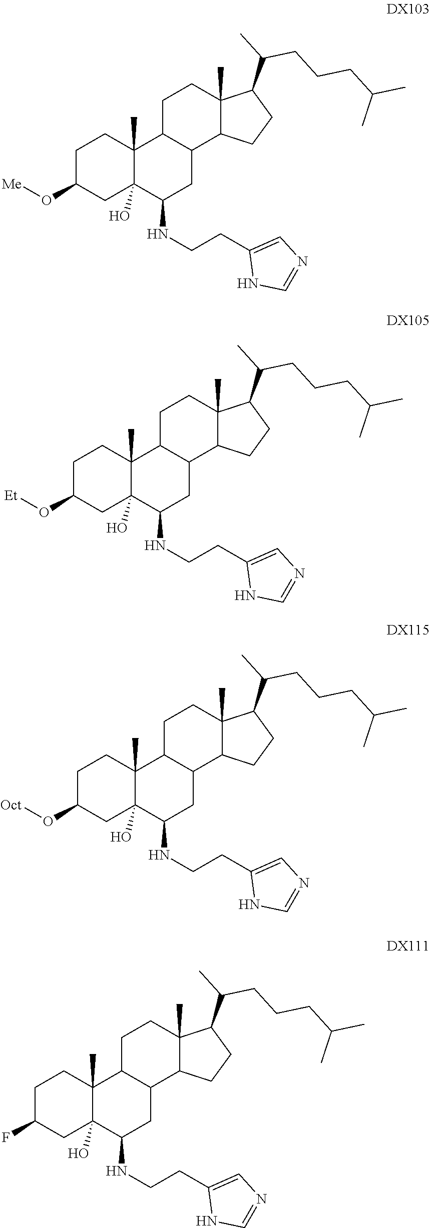

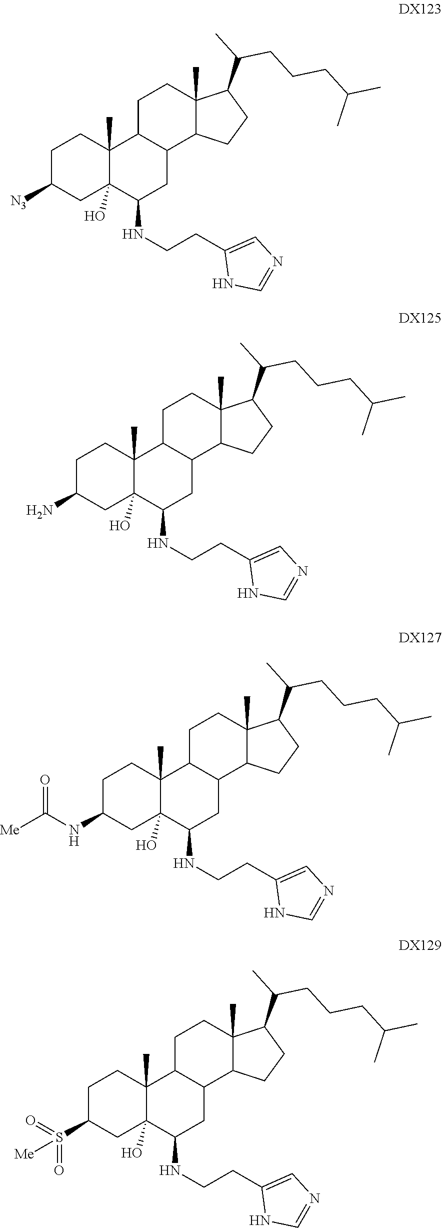

The other compounds falling within the scope of the general formula, not described, form an integral part of the compounds according to the invention.

### Example 1: Synthesis of the Analog Compound 3β-fluoro-5α-hydroxy-6β-[2-(1H-imidazol-4-yl)ethylamino]cholestane (named DX111)

The first step is a synthesis of the compound 3β-fluorocholestane comprising the following steps:

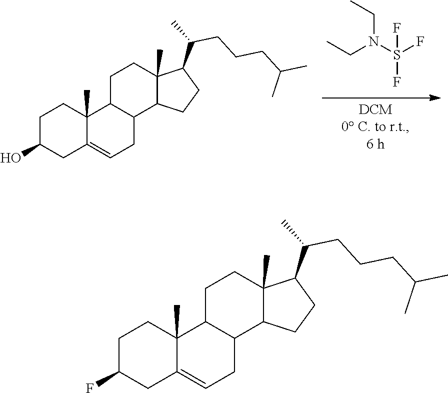

5.00 g of diethylaminosulfur trifluoride (d=1.22 g/ml, 31.0 mmol) were dissolved in 200 ml of anhydrous DCM. 6.66 grams (g) of cholesterol (17.2 mmol) were dissolved in 100 milliliters (ml) of anhydrous dichloromethane and added dropwise to the fluoro reagent at 0° C. The mixture thus obtained was left under magnetic stirring for 5 hours, while allowing it to warm up to room temperature. After this period, the reaction was neutralized by adding 100 ml of saturated NaHCO₃ solution. The mixture was transferred into a separating funnel and the organic phase was washed twice with saturated NaHCO₃, twice more with saturated NaCl solution and once with water. The organic phase was dried over MgSO₄, filtered and then evaporated to obtain a white powder. 6.61 g corresponding to 3β-fluorocholestane were obtained. The final reaction yield is 99%.

¹H-NMR (500 MHz, CDCl₃): δ (ppm) 5.40-5.39 (d, 1H), 4.47-4.30 (m, 1H), 2.45-2.42 (t, 2H), 2.03-1.95 (m, 3H), 1.90-0.95 (m, 26H), 0.92-0.91 (d, 3H), 0.87-0.85 (dd, 6H), 0.68 (s, 3H).

The second step consists in synthesizing, starting with 3β-fluorocholestane, the compound 3β-fluoro-5,6α-epoxycholestane as follows:

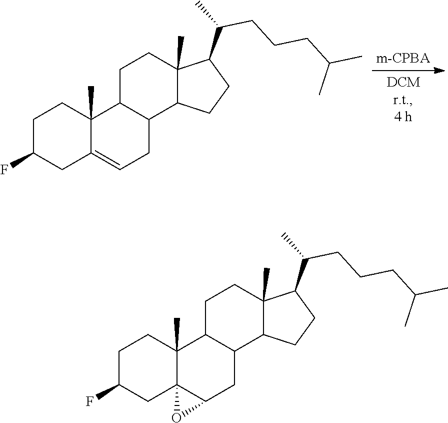 [SMILES: CC(C)CCC[C@@H](C)[C@H]1CCC2C3CC=C4C[C@@H](F)CC[C@]4(C)C3CC[C@@]21C ; CC(C)CCC[C@@H](C)[C@H]1CCC2C3C[C@@H]4O[C@@]45C[C@@H](F)CC[C@]5(C)C3CC[C@@]21C]

4.96 g of meta-chloroperoxybenzoic acid at 77% purity (22.1 mmol) were dissolved in 100 ml of dichloromethane and added dropwise to a mixture of 6.61 g of 3β-fluorocholestane (17.0 mmol) dissolved in 50 ml of dichloromethane. The mixture thus obtained was stirred and maintained at room temperature for 3 hours. The mixture obtained was washed twice with an aqueous solution containing 10% by weight of Na₂S₂O₃, twice with saturated NaHCO₃ solution and with saturated NaCl solution. The organic phase was dried over anhydrous MgSO₄. Vacuum evaporation of the organic solvent was performed to give 6.90 g of a white powder comprising: 3β-fluoro-5,6α-epoxycholestane (85% of the white powder) and 3β-fluoro-5,6β-epoxycholestane (15% of the white powder). The 3β-fluoro-5,6α-epoxycholestane was used without further purification.

¹H-NMR (500 MHz, CDCl₃): δ (ppm) 4.82-4.64 (m, 1H), 2.91-2.90 (d, 1H), 2.28-2.21 (m, 1H), 2.10-2.06 (m, 1H), 1.97-1.70 (m, 6H), 1.59-0.92 (m, 23H), 0.89-0.88 (d, 3H), 0.87-0.85 (dd, 6H), 0.61 (s, 3H).

The third consists in synthesizing 3β-fluoro-5α-hydroxy-6β-[2-(1H-imidazol-4-yl)ethylamino]cholestane (DX111 in basic form) as follows:

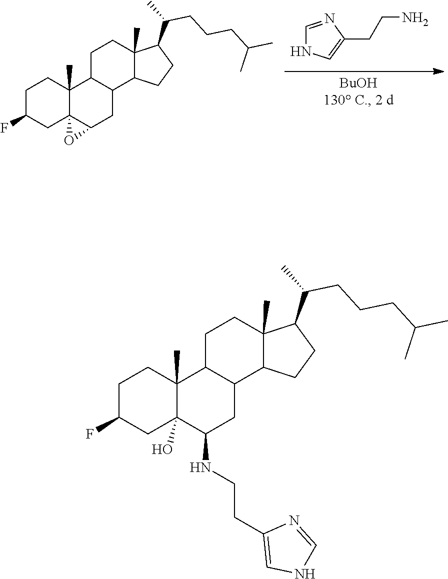

0.80 g of histamine in its basic form (7.2 mmol) was added to a 10 ml butanolic solution comprising 1.45 g of the compound 3β-fluoro-5,6α-epoxycholestane (3.6 mmol) with stirring at 130° C. The mixture was kept stirring at reflux, heating at a temperature of 130° C. for 48 hours.

The reaction progress can be monitored by thin layer chromatography (TLC) to follow the conversion of the 3β-fluoro-5,6α-epoxycholestane.

After cooling, the mixture was diluted in 15 ml of methyl tert-butyl ether. The organic phase was washed with 3 times 15 ml of water.

The organic phase was dried over anhydrous MgSO₄, filtered and then evaporated to give a brown oil. The mixture was purified by column chromatography on silica gel on a purification machine comprising a 20 g prepacked column, eluting with 100% ethyl acetate. A white powder of 0.86 g of 3β-fluoro-5α-hydroxy-6β-[2-(1H-imidazol-4-yl)ethylamino]cholestane was obtained. The final reaction yield was 41% with a purity of greater than 97% measured by NMR (nuclear magnetic resonance) and TLC (thin layer chromatography) analysis.

¹H-NMR (500 MHz, CDCl₃): δ (ppm) 7.54 (s, 1H), 6.80 (s, 1H), 5.05-4.88 (m, 1H), 3.03-2.96 (m, 1H), 2.77-2.73 (m, 3H), 2.46 (s, 1H), 2.27-2.20 (q, 1H), 2.00-1.98 (d, 2H), 1.86-0.94 (m, 31H), 0.91-0.89 (d, 3H), 0.87-0.85 (d, 6H), 0.67 (s, 3H).

### Example 2: Preparation of a Dilactate Salt of the Compound 3β-fluoro-5α-hydroxy-6β-[2-(1H-imidazol-4-yl)ethylamino]cholestane (DX111 in dilactate form)

A dilactate salt of the compound 3β-fluoro-5α-hydroxy-6β-[2-(1H-imidazol-4-yl)ethylamino]cholestane was prepared in the following manner:

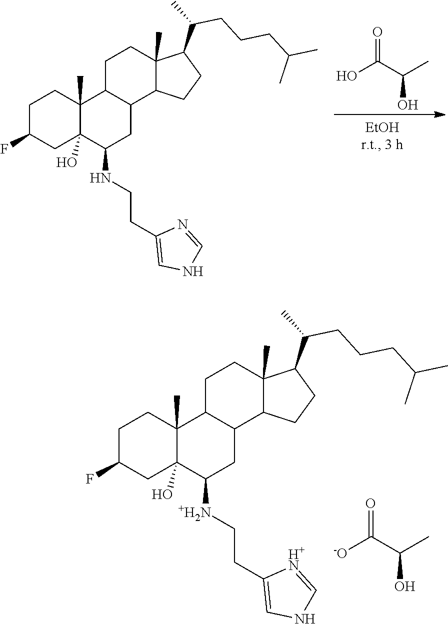

267.2 mg of lactic acid (2.97 mmol) were added to a solution of 0.76 g of 3β-fluoro-5α-hydroxy-6β-[2-(1H-imidazol-4-yl)ethylamino]cholestane in 15 ml of anhydrous ethanol with stirring.

Stirring was continued at room temperature for 3 hours. Vacuum evaporation of the organic solvent gave a white powder of 1.03 g of 3β-fluoro-5α-hydroxy-6β-[2-(1H-imidazol-4-yl)ethylamino]cholestane dilactate.

1H-NMR (500 MHz, MeOD-4d): δ (ppm) 7.58 (s, 1H), 6.79 (s, 1H), 4.73-4.57 (m, 1H), 3.86-3.82 (dd, 2H), 3.35-3.31 (dd, 2H), 3.18-3.13 (m, 1H), 3.03-2.98 (m, 1H), 2.77-2.75 (t, 2H), 2.70 (s, 1H), 2.12-2.05 (dd, 1H), 1.78-1.76 (d, 1H), 1.70-1.68 (d, 1H), 1.63-0.85 (m, 30H), 0.78-0.73 (d, 2H), 0.68-0.66 (d, 3H), 0.61-0.60 (dd, 6H), 0.49 (s, 3H).

### Example 3: Preparation of the 3α-Amino and 3α-Sulfide Derivatives or Analogs of Formula (I)

The steps are as follows:

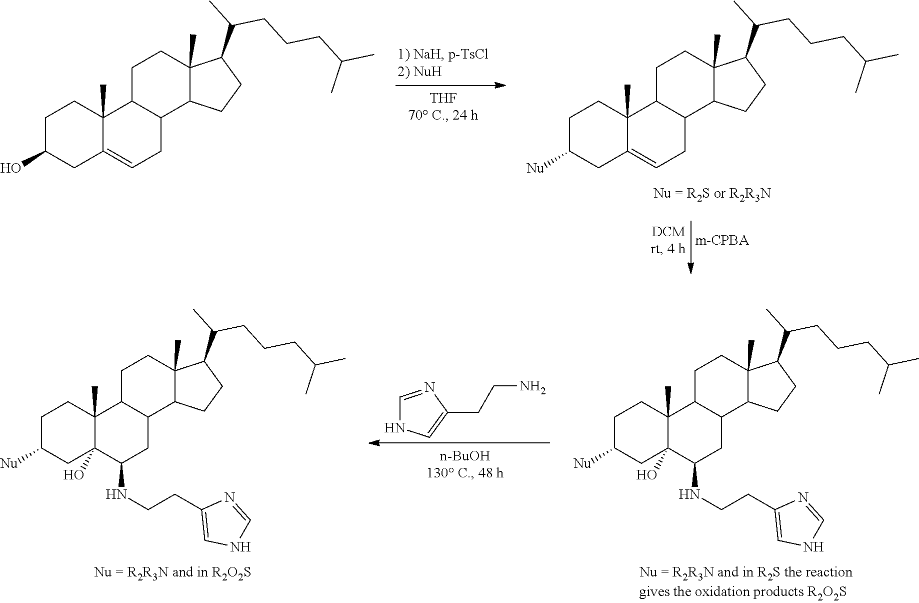

Stir cholesterol in tetrahydrofuran (THF) in the presence of NaH for five minutes at 70° C., add para-toluenesulfonyl chloride (p-TsCI) and stir the mixture for 4 hours at 70° C. Add water, filter the reaction mixture and evaporate off the organic solvents. Extract the reaction product with dichloromethane/water (DCM/H₂O) and dry over MgSO₄. Remove the organic solvents by vacuum evaporation. The product obtained is used as is for the next step. Dissolve the product obtained in THF with stirring for 12 hours with 1.1 equivalents of NuH (Nucleophile-Hydrogen). NuH corresponds to R₂SH or NHR₂R₃ at 70° C. The reaction is quenched by addition of water and the products were extracted with an EtOAc/H₂O system. The organic phase was dried over MgSO₄ and the organic solvents were evaporated off under vacuum. The cholestane 3-sulfide and cholestane 3-amino derivatives are purified either by column chromatography or by recrystallization. The reaction pathways to obtain the dendrogenin A analogs are the same steps developed for the synthesis of dendrogenin A.

The product R₂O₂S will be obtained by oxidation of R₂S with oxidizing agents such as m-CPBA or H₂O₂.

### Example 4: Preparation of 3β Amino and 3β Sulfide Derivatives or Analogs of Formula (I)

### The Steps are Shown Below:

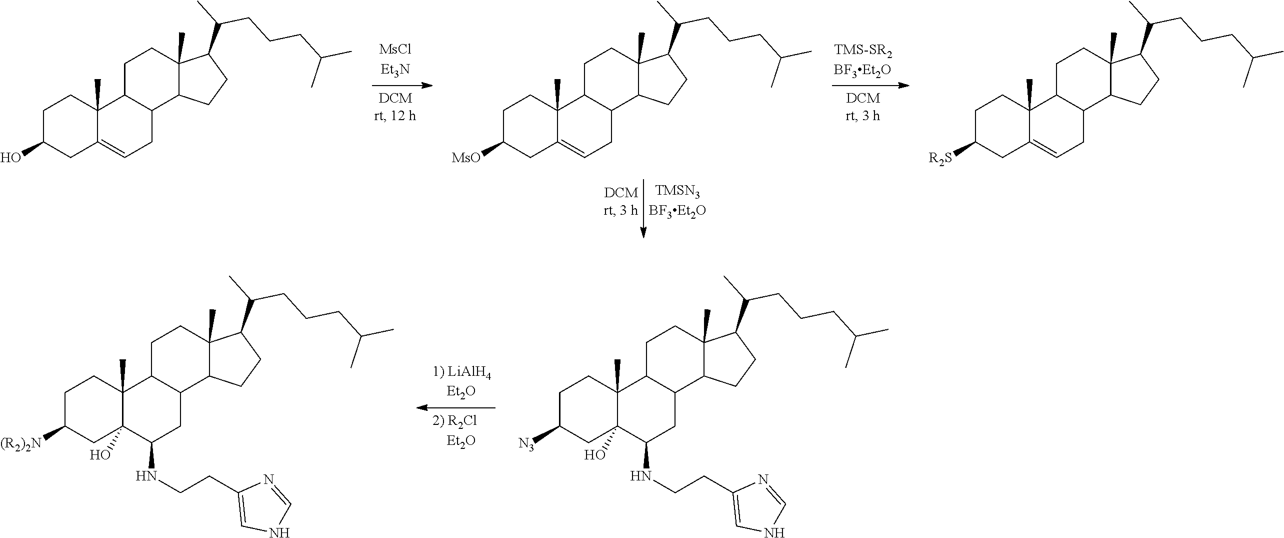

The steps are shown below: Dissolve cholesterol, add Et₃N in DCM and add mesyl chloride (MsCI) dropwise in DCM solution at room temperature over 1 h. Stir the reaction mixture for 12 h, then evaporate off the organic solvent and crystallize the product from MeOH. The product obtained is a white solid. The product obtained is used to obtain the 3β-sulfide and 3β-azide derivatives. The obtained product is dissolved in DCM and TMS-SR₂ for the 3β-sulfide derivative or TMS-N₃ for the 3β-azide derivative is then added to the solution. Addition of BF₃*Et₂O is performed at room temperature. The mixture is then stirred for 3 h.

The 3β-azide is reduced to 3β-amino by the action of LiAIH₄ in Et₂O and transformed into the products of formula (I) with reaction of R₂X (X═Br, Cl or 1) in Et₂O (or pyridine) as solvent. The reaction pathways to obtain the dendrogenin A analogs are the same steps developed for the synthesis of dendrogenin A. The sulfonyl derivative R₂O₂S will be obtained by oxidation of R₂S with usual oxidizing agents. This method is detailed in the literature: Organic Letters, 2009, 11, 3, 567-570, “Practical Synthesis of 3β-Amino-5-cholestene and Related 3, β-Halides Involving i-Steroid and Retro-i-Steroid Rearrangements” (https://doi.org/10.1021/ol802343z).

### Example 5: Cytotoxicity Study of 3β-fluoro-5α-hydroxy-6β-[2-(1H-imidazol-4-yl)ethylamino]cholestane (Named DX111)

For this experiment, a cell culture medium was prepared. The culture medium consisted of Dulbecco's Modified Eagle Medium (DMEM, sold by Westburg under the reference LO BE12-604F), comprising 4.5 g/L glucose with L-glutamine, to which 10% fetal calf serum (FCS) is added. Neuro2a (murine neuroblastoma) cells are introduced into this culture medium.

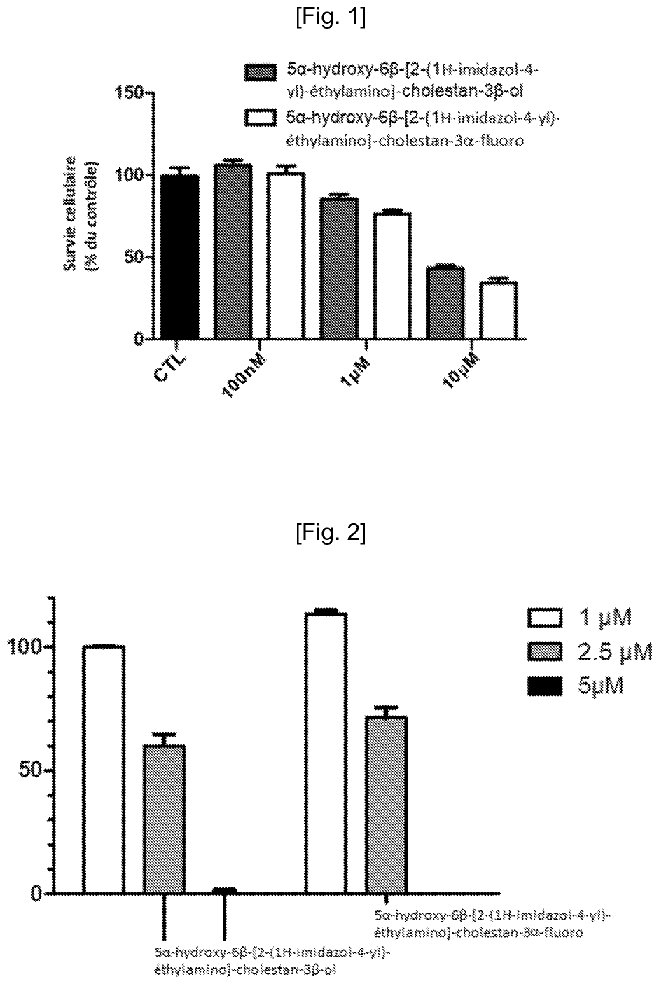

24-well dishes were seeded with 10 000 Neuro2a cells per well. After 72 hours (h) of culture under normal conditions, i.e., in an incubator at 37° C. with 5% CO₂, the Neuro2a cells were treated for 48 h with 3β-fluoro-5α-hydroxy-6β-[2-(1H-imidazol-4-yl)ethylamino]-cholestane and 5α-hydroxy-6β-[2-(1H-imidazol-4-yl)ethylamino]cholestan-3β-ol at 100 nM, 1 μM and 10 μM. A control (CTL) is also performed using the previously described protocol without treatment with 3α-fluoro-5α-hydroxy-6β-[2-(1H-imidazol-4-yl)ethylamino]-cholestane and 5α-hydroxy-6β-[2-(1H-imidazol-4-yl)ethylamino]cholestan-3β-ol. The cell survival was quantified by means of a trypan blue test with automatic counting using the Biorad TC20 machine (TC20™ Automated Cell Counter). The trypan blue test is based on the integrity of cell membranes, which is disrupted in the dead cells. Trypan blue stains dead cells blue. The Biorad TC20 cell counter counts the proportion of blue and non-blue cells, and reports the percentage of cells. The results are represented in FIG. 1. FIG. 1 shows on the y-axis the percentage of cell survival relative to the control group.

It is illustrated in FIG. 1 that for 100 nM of 3β-fluoro-5α-hydroxy-6β-[2-(1H-imidazol-4-yl)ethylamino]cholestane treatment, the percentage of live cells remains unchanged compared to the control group (CTL). Furthermore, for concentrations of 1 μM and 10 μM, the percentage of cell survival is 75% and 30%. Similar activity is also observed between the two test compounds. In conclusion, cytotoxic activity of the compound 3β-fluoro-5α-hydroxy-6β-[2-(1H-imidazol-4-yl)ethylamino]cholestane of formula (I) is observed toward Neuro2a tumor cells for 3α-fluoro-5α-hydroxy-6β-[2-(1H-imidazol-4-yl)ethylamino]cholestane concentrations of 1 μM and 10 μM.

### Example 6: Effect of 3β-fluoro-5α-hydroxy-6β-[2-(1H-imidazol-4-yl)ethylamino]-Cholestane on the Viability of MCF-7 Cells

A cell viability test was performed on MCF-7 (Michigan Cancer Foundation-7) breast tumor cells overexpressing HER2 (ER(+) cells).

The MCF-7 cells are in a cell culture medium identical to that of Example 5 and are seeded in 12-well plates at 50 000 cells per well. 24 hours after seeding, the cells are treated with vehicle solvate comprising water and ethanol with a 1% o ratio of ethanol, 3β-fluoro-5α-hydroxy-6β-[2-(1H-imidazol-4-yl)ethylamino]cholestane, and 5α-hydroxy-6β-[2-(1H-imidazol-4-yl)ethylamino]cholestan-3β-ol at 1, 2.5 or 5 μM. The cells are observed under an inverted microscope and photographed via the microscope camera at 24 h and 48 h. The morphological changes in the cells at 1 μM are very small. Only a few white vesicles are observed, reflecting the start of the autophagy phenomenon, giving rise to cell death after 24 h of treatment with 3β-fluoro-5α-hydroxy-6β-[2-(1H-imidazol-4-yl)ethylamino]-cholestane and 5α-hydroxy-6β-[2-(1H-imidazol-4-yl)ethylamino]cholestan-3β-ol. The effects are more marked at 2.5 μM and 5 μM with an increase in the number of dead cells. Indeed, numerous white vesicles and detached cells are observed after 24 h of treatment with 3β-fluoro-5α-hydroxy-6β-[2-(1H-imidazol-4-yl)ethylamino]cholestane at 2.5 μM. After 24 h of treatment with 3β-fluoro-5α-hydroxy-6β-[2-(1H-imidazol-4-yl)ethylamino]-cholestane at 5 μM, 99% of the observed cells are supernatant, reflecting cell death, and 1% of the cells are adherent and show white vesicles. After 48 h of treatment with 3β-fluoro-5α-hydroxy-6β-[2-(1H-imidazol-4-yl)ethylamino]cholestane at 2.5 μM, a stronger cytostatic effect is observed than at 24 h and more cells are rounding, reflecting cell death. The cytostatic effect is illustrated by an inhibition of cell proliferation. After 48 h of treatment with 3β-fluoro-5α-hydroxy-6β-[2-(1H-imidazol-4-yl)ethylamino]cholestane at 5 μM, all the cells are supernatant. Compared with treatment with 5α-hydroxy-6β-[2-(1H-imidazol-4-yl)ethylamino]cholestan-3β-ol, treatment with 3β-fluoro-5α-hydroxy-6β-[2-(1H-imidazol-4-yl)ethylamino]cholestane shows a greater or equal effect after 24 h of observation and similar after 48 h of observation.

The cell viability is measured by labeling with MTT at 48 hours. This test is based on the use of the tetrazolium salt MTT (3-(4,5-dimethylthiazol-2-yl)-2,5-diphenyltetrazolium bromide). Tetrazolium is reduced by mitochondrial succinate dehydrogenase in active living cells to formazan, a purple colored precipitate. The amount of precipitate formed is proportional to the amount of living cells but also to the metabolic activity of each cell. Thus, a simple determination of the optical density at 540 nm by spectroscopy makes it possible to determine the relative amount of living and metabolically active cells. After 48 hours, the medium is aspirated, and the cells are washed with phosphate-buffered saline (PBS) and then incubated with MTT (0.5 mg/ml in PBS) for about 2 hours. The MTT solution is aspirated and the purple crystals are dissolved in dimethyl sulfoxide (DMSO). The OD (optical density) is measured at 540 nm.

The results of this test are shown in FIG. 2. FIG. 2 shows on the y-axis the percentage of cell viability relative to the control group. The control group is prepared in a similar manner to the groups studied without the addition of the molecules studied in the present text. Compared with the control, a dose-dependent decrease in cell viability in MTT is measured for 3β-fluoro-5α-hydroxy-6β-[2-(1H-imidazol-4-yl)ethylamino]cholestane and 5α-hydroxy-6β-[2-(1H-imidazol-4-yl)ethylamino]cholestan-3β-ol. For a concentration of 5 μM, the viability is close to 0%. This reflects the ability of the compound of formula (I) to kill mammary tumor cells. These results are consistent with the abovementioned observations made at 24 h and 48 h.

### Example 7: Effect of 3β-fluoro-5α-hydroxy-6β-[2-(1H-imidazol-4-yl)ethylamino]-Cholestane on Cholesterol Epoxide Hydrolase (ChEH) Activity in MCF-7 Cells

The compounds 5,6α-epoxycholesterol (5,6α-EC) and 5,6p-epoxycholesterol (5,6p-EC) are oxysterols involved in the anticancer pharmacology of tamoxifen, a widely used antitumor drug. Both are metabolized to cholestane-3β,5α,6β-triol (CT) by the enzyme cholesterol-5,6-epoxide hydrolase (ChEH), and CT is metabolized by the enzyme HSD11B2 (11β-hydroxysteroid dehydrogenase 2) to 6-oxocholestane-3β,5α-diol (OCDO), a tumor-promoting oncosterone.

The purpose of the following experiment is to demonstrate the ability of 3α-fluoro-5α-hydroxy-6β-[2-(1H-imidazol-4-yl)ethylamino]cholestane to block ChEH and thus limit the metabolization of oncosterone, a tumor-promoting metabolite.

MCF-7 cells are in a cell culture medium identical to that of Example 5 and are seeded in 6-well plates at 150 000 cells per well with three wells per treatment condition. 24 h after seeding, the MCF-7 cells are treated with [¹⁴C]5,6α-EC (1000X stock solution: 0.6 mM; 20 μCi/μmol; final concentration 0.6 μM) alone or in combination with tamoxifen (tam). Tamoxifen is used as a positive control for 3α-fluoro-5α-hydroxy-6β-[2-(1H-imidazol-4-yl)ethylamino]cholestane and 5α-hydroxy-6β-[2-(1H-imidazol-4-yl)ethylamino]cholestan-3β-ol (1 μM for all molecules).

After 24 hours of treatment, the media are collected and lipid extracts are prepared from the cell pellets by extraction with 100 μL of chloroform, 400 μL of methanol, and 300 μL of water.

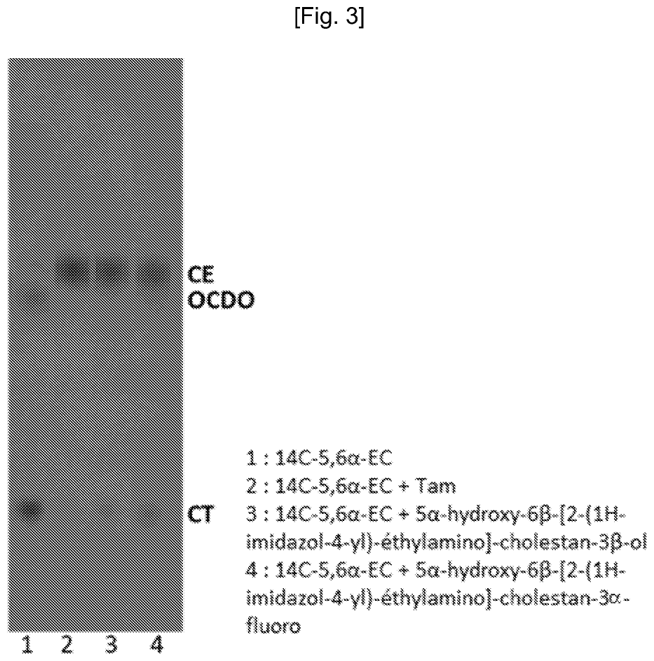

The lipid extracts are analyzed by thin layer chromatography (TLC) using ethyl acetate (EtOAc) as eluent. The analysis is performed with a plate reader and then by autoradiography. The results are presented in FIG. 3. Almost total metabolism of the epoxide to CT and OCDO is observed (wells 2 and 4) and total inhibition of ChEH activity by tamoxifen and almost total inhibition (trace of CT) by 3α-fluoro-5α-hydroxy-6β-[2-(1H-imidazol-4-yl)ethylamino]cholestane. Similar results are observed with 5α-hydroxy-6β-[2-(1H-imidazol-4-yl)ethylamino]cholestan-3β-ol.

In conclusion, 3α-fluoro-5α-hydroxy-6β-[2-(1H-imidazol-4-yl)ethylamino]cholestane has ChEH-inhibiting activity similar to that of 5α-hydroxy-6β-[2-(1H-imidazol-4-yl)ethylamino]cholestan-3β-ol.

### Example 8: Synthesis of the Compound of formula (I) 3β-methoxy-5α-hydroxy-6β-[2-(1H-imidazol-4-yl)ethylamino]cholestane (named DX103)

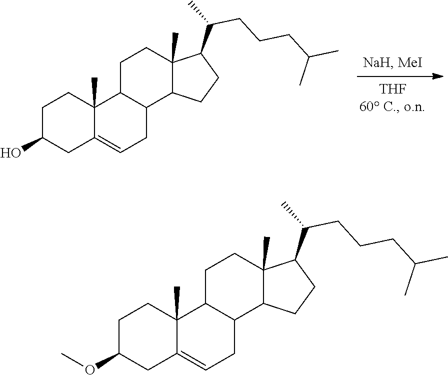

A first step consists in dissolving 4.0 grams (g) of cholesterol (10.3 mmol) in 20 milliliters (ml) of tetrahydrofuran (THF). 0.80 g of NaH (60% in oil, 20.0 mmol) was added and allowed to react for 30 minutes at 60° C., then 1.8 ml of iodomethane (28.9 mmol) were added. The mixture thus obtained was left at 60° C. overnight, i.e. about 10 h. After cooling the solution, the reaction was neutralized by adding 20 ml of water. The mixture was filtered and the THF evaporated off under vacuum. The mixture was transferred into a separating funnel and the aqueous phase was extracted three times with ethyl acetate. The resulting organic phases were combined and dried over MgSO₄ and then evaporated to give an oil. The obtained oil was dissolved in 2 ml of Et₂O and MeOH was added until a white precipitate was formed. The powder was filtered off, washed with cold MeOH and dried. A white powder of 3.40 g (corresponding to a yield of 82%) of 3β-methoxycholestane was thus obtained.

¹H-NMR (500 MHz, CDCl₃): δ (ppm) 5.36 (s, 1H), 3.35 (s, 3H), 3.09-3.02 (q, 1H), 2.40-2.36 (d, 1H), 2.18-2.13 (t, 1H), 2.03-1.81 (m, 5H), 1.60-1.00 (m, 24H), 0.92-0.91 (d, 3H), 0.87-0.85 (dd, 6H), 0.68 (s, 3H).

The second step consists in synthesizing, starting with 3β-methoxycholestane, the compound 3β-methoxy-5,6α-epoxycholestane as follows:

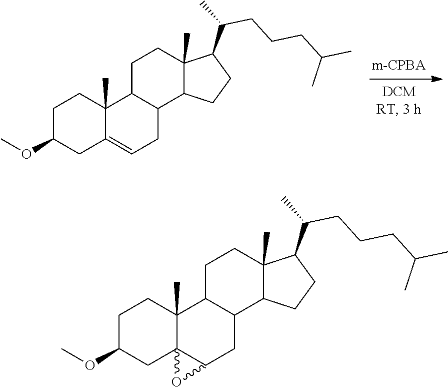

1.80 g of meta-chloroperoxybenzoic acid (8.90 mmol) were dissolved in 70 ml of dichloromethane and added dropwise to a mixture of 2.50 g of 3β-methoxycholestane (6.24 mmol) dissolved in 20 ml of dichloromethane. The mixture thus obtained was stirred and maintained at room temperature for three hours. The mixture thus obtained was washed with an aqueous solution containing 10% by weight of Na₂S₂O₃, saturated NaHCO₃ solution and saturated NaCl solution. The organic phase was dried over anhydrous MgSO₄. Vacuum evaporation of the organic solvent was performed, to give a transparent viscous oil. 5 ml of Et₂O were added to dissolve the oil, 25 ml of EtOH were then added and the mixture was heated to the boiling point three times and finally maintained at 0° C. overnight to promote precipitation. A white powder was filtered off, washed with cold MeOH and dried; 1.73 g, corresponding to a yield of 67% (with an enantiomeric excess 90%), of 3β-methoxy-5,6α-epoxycholestane were thus obtained.

¹H-NMR (500 MHz, CDCl₃): δ (ppm) 3.45-3.39 (m, 1H), 3.33 (s, 3H), 2.90-2.89 (d, 1H), 2.00-0.94 (m, 31H), 0.89-0.88 (d, 3H), 0.86-0.85 (dd, 6H), 0.60 (s, 3H).

The third step consists in synthesizing 3β-methoxy-5α-hydroxy-6β-[2-(1H-imidazol-4-yl)ethylamino]cholestane (DX103 basic form) as follows:

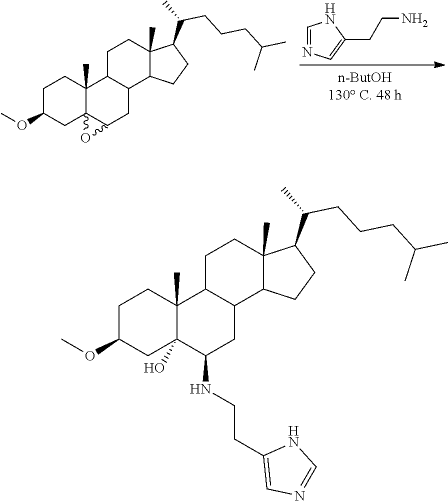

0.81 g of histamine (7.30 mmol) in its basic form was added to a 10 ml butanolic solution comprising 1.50 g of the compound 3β-methoxy-5,6α-epoxycholestane (3.62 mmol) with stirring. The mixture was kept stirring at reflux, heating at a temperature of 130° C. for 48 hours. The reaction progress can be monitored by thin layer chromatography (TLC) to follow the conversion of the 3β-methoxy-5,6α-epoxycholestane.

After cooling, the mixture was diluted in 10 ml of methyl tert-butyl ether. The organic phase was washed twice with 10 ml of water and then once with 10 ml of saturated NaCl solution.

The organic phase was dried over anhydrous MgSO₄. The mixture was purified by column chromatography on a purification machine. The eluent used was a 90%/10% mixture of ethyl acetate and methanol. A white powder of 1.32 g of 3β-methoxy-5α-hydroxy-6β-[2-(1H-imidazol-4-yl)ethylamino]cholestane was obtained. The final reaction yield was 69% with a purity of greater than 95% measured by NMR (nuclear magnetic resonance) and TLC (thin layer chromatography) analysis.

¹H-NMR (500 MHz, MeOD-4d): δ (ppm) 7.62 (s, 1H), 6.88 (s, 1H), 3.71-3.65 (m, 1H), 3.34 (s, 3H), 2.98-2.97 (d, 1H), 2.78-2.77 (m, 3H), 2.45 (s, 1H), 2.03-2.00 (m, 1H), 1.94-1.83 (m, 3H), 1.65-1.01 (m, 27H), 0.95-0.94 (d, 3H), 0.91-0.89 (d, 6H), 0.71 (s, 3H).

### Example 9: Preparation of a Dilactate Salt of the Compound 3β-methoxy-5α-hydroxy-6β-[2-(1H-imidazol-4-yl)ethylamino]cholestane (DX103 dilactate form)

A dilactate salt of the compound 3β-methoxy-5α-hydroxy-6β-[2-(1H-imidazol-4-yl)ethylamino]cholestane was prepared as follows:

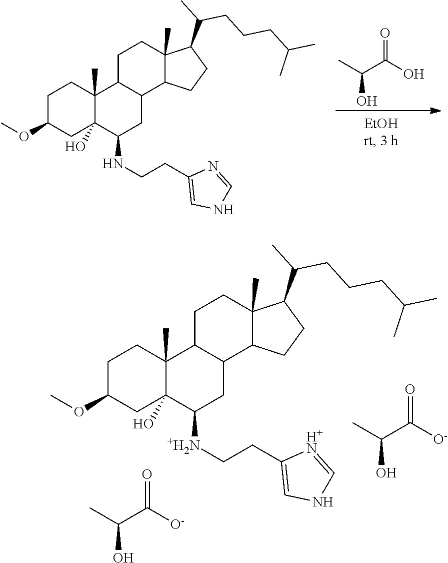

21.0 mg of lactic acid (1.89 mmol) were added to a solution of 0.50 g of 3β-methoxy-5α-hydroxy-6β-[2-(1H-imidazol-4-yl)ethylamino]cholestane (0.95 mmol) in 15 ml of anhydrous ethanol with stirring. Stirring was continued at room temperature for 3 hours. Vacuum evaporation of the organic solvent afforded a white powder of 0.52 g of 3β-methoxy-5α-hydroxy-6β-[2-(1H-imidazol-4-yl)ethylamino]cholestane dilactate.

¹H-NMR (500 MHz, MeOD-4d): δ (ppm) 7.61 (s, 1H), 6.84 (s, 1H), 3.93-3.89 (q, 2H), 3.62-3.57 (m, 1H), 3.39-3.09 (m, 8H), 3.21-3.16 (m, 1H), 2.84-2.74 (m, 3H), 1.91-1.81 (m, 2H), 1.70-0.79 (m, 31H), 0.73-0.72 (d, 3H), 0.68-0.66 (d, 6H), 0.56 (s, 3H).

### Example 10: Synthesis of the Compound of Formula (I) 3β-Ethoxy-5α-Hydroxy-6β-[2-(1H-imidazol-4-yl)ethylamino]cholestane (named DX105)

The first step is a synthesis of the compound 3β-ethoxycholestane comprising the following steps:

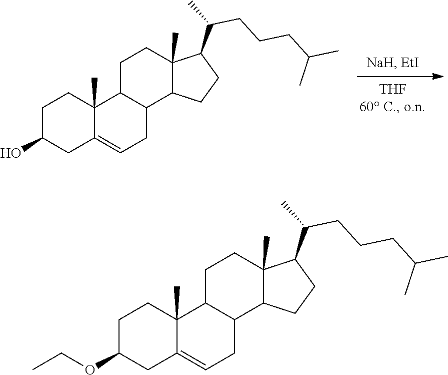

4.00 g of cholesterol (10.3 mmol) were dissolved in 20 ml of THF. 0.82 g of NaH (60% in oil, 20.0 mmol) was added and allowed to react for 30 minutes at 60° C., then 1.9 ml of iodoethane (28.9 mmol) were added. The mixture thus obtained was left at 60° C. overnight. After cooling the solution, the reaction was neutralized by addition of 20 ml of water. The mixture was filtered and the THF evaporated off under vacuum. The mixture was transferred into a separating funnel and the aqueous phase was extracted three times with ethyl acetate. The organic phases thus obtained were combined and dried over MgSO₄ and then evaporated to give an oil. The oil thus obtained was dissolved in 2 ml of Et₂O and MeOH was added until a white precipitate was formed. The powder was filtered off, washed with cold MeOH and dried. A white powder of 2.12 g (corresponding to a 49% yield) of 3β-ethoxycholestane was thus obtained.

¹H-NMR (500 MHz, CDCl₃): δ (ppm) 5.35 (s, 1H), 3.53-3.51 (q, 2H), 3.17-3.14 (m, 1H), 2.38-2.35 (d, 1H), 2.22-2.17 (t, 3H), 2.02-1.79 (m, 5H), 1.60-0.94 (m, 27H), 0.92-0.91 (d, 3H), 0.87-0.85 (dd, 6H), 0.67 (s, 3H).

The second step consists in synthesizing, starting with 3β-ethoxycholestane, the compound 3β-ethoxy-5,6α-epoxycholestane as follows:

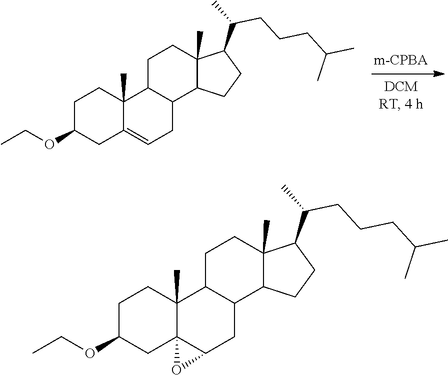

1.44 g of meta-chloroperoxybenzoic acid (corresponding to 6.43 mmol) were dissolved in 50 ml of dichloromethane and added dropwise to a mixture of 2.0 g of 3β-ethoxycholestane (4.82 mmol) dissolved in 10 ml of dichloromethane. The mixture thus obtained was stirred and maintained at room temperature for 3 hours. The mixture thus obtained was washed with an aqueous solution containing 10% by weight of Na₂S₂O₃, saturated NaHCO₃ solution and saturated NaCl solution. The organic phase was dried over anhydrous MgSO₄. Vacuum evaporation of the organic solvent was performed, to give a transparent viscous oil. 5 ml of Et₂O were added to dissolve the oil, then 25 ml of EtOH were added and the mixture was heated to the boiling point three times and then maintained at 0° C. overnight to promote precipitation. A white powder was filtered off, washed with cold MeOH and dried: 0.72 g, corresponding to a yield of 35% (with an enantiomeric excess 90%) of 3β-ethoxy-5,6α-epoxycholestane was thus obtained.

¹H-NMR (500 MHz, CDCl₃): δ (ppm) 3.55-3.46 (m, 3H), 2.89-2.88 (d, 1H), 2.04-0.93 (m, 34H), 0.89-0.88 (d, 3H), 0.86-0.85 (dd, 6H), 0.60 (s, 3H).

The third step consists in synthesizing 3β-ethoxy-5α-hydroxy-6β-[2-(1H-imidazol-4-yl)ethylamino]cholestane (DX105 in basic form) as follows:

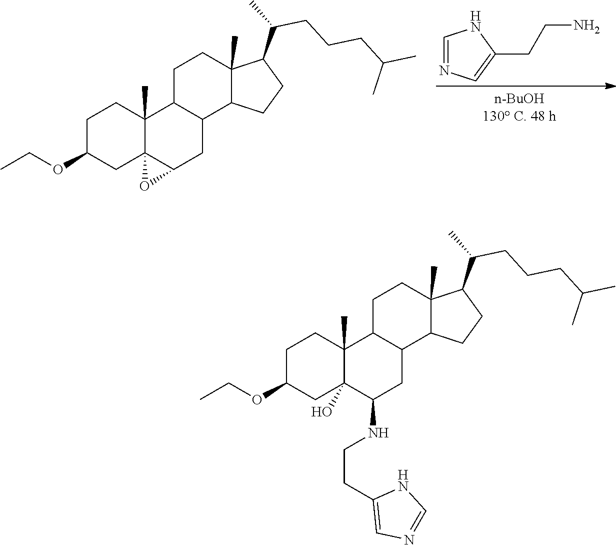

0.31 g of histamine in its basic form (correspond to 2.74 mmol) was added to a 5 ml butanolic solution comprising 0.51 g of the compound 3β-ethoxy-5,6α-epoxycholestane (1.18 mmol) with stirring. The mixture was kept stirring at reflux, heating at a temperature of 130° C. for 48 hours.

The reaction progress can be monitored by thin layer chromatography (TLC) to follow the conversion of the 3β-ethoxy-5,6α-epoxycholestane.

After cooling, the mixture was diluted in 5 ml of methyl tert-butyl ether. The organic phase was washed twice with 5 ml of water and then once with 5 ml of saturated NaCl solution.

The organic phase was dried over anhydrous MgSO₄. The mixture was purified by column chromatography on a purification machine. The eluent used was a 90/10 ethyl acetate/methanol mixture. A white powder of 0.28 g of 3β-ethoxy-5α-hydroxy-6β-[2-(1H-imidazol-4-yl)ethylamino]cholestane was obtained. The final reaction yield was 44% with a purity of greater than 97% measured by NMR (nuclear magnetic resonance) and TLC (thin layer chromatography) analysis.

¹H-NMR (500 MHz, MeOD-4d): δ (ppm) 7.62 (s, 1H), 6.89 (s, 1H), 3.82-3.76 (m, 1H), 3.57-3.52 (q, 2H), 3.05-3.00 (m, 1H), 2.85-2.80 (m, 3H), 2.50 (s, 1H), 2.03-1.83 (m, 5H), 1.65-1.51 (m, 7H), 1.42-1.01 (m, 22H), 0.96-0.94 (d, 3H), 0.91-0.89 (d, 6H), 0.72 (s, 3H).

### Example 11: Preparation of a Dilactate Salt of the Compound 3β-ethoxy-5α-hydroxy-6β-[2-(1H-imidazol-4-yl)ethylamino]cholestane (DX105 in Dilactate Form)

A dilactate salt of the compound 3β-ethoxy-5α-hydroxy-6β-[2-(1H-imidazol-4-yl)ethylamino]cholestane was prepared in the following manner:

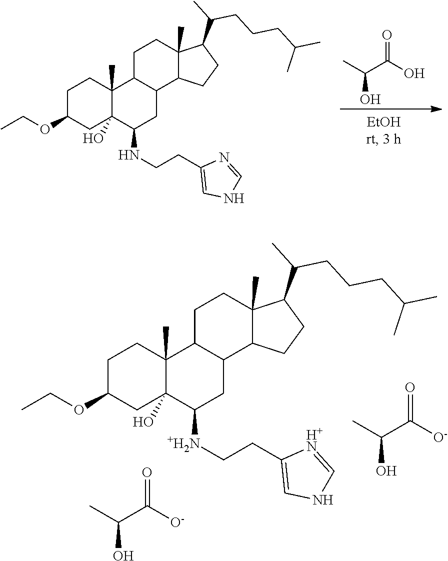

166.2 mg of lactic acid (1.85 mmol) were added to a solution of 0.50 g of 3β-ethoxy-5α-hydroxy-6β-[2-(1H-imidazol-4-yl)ethylamino]cholestane (0.92 mmol) in 5 ml of anhydrous ethanol with stirring. Stirring was continued at room temperature for 3 hours. Vacuum evaporation of the organic solvent gave a white powder of 0.20 g of 3β-ethoxy-5α-hydroxy-6β-[2-(1H-imidazol-4-yl)ethylamino]cholestane dilactate.

¹H-NMR (500 MHz, MeOD-4d): δ (ppm) 7.61 (s, 1H), 6.84 (s, 1H), 3.92-3.89 (q, 2H), 3.60-3.57 (m, 1H), 3.39-3.09 (m, 7H), 2.84-2.74 (m, 3H), 1.91-1.81 (m, 2H), 1.70-0.79 (m, 34H), 0.73-0.72 (d, 3H), 0.67-0.65 (d, 6H), 0.54 (s, 3H).

### Example 12: Synthesis of the Compound of Formula (I) 3β-octanoxy-5α-hydroxy-6β-[2-(1H-imidazol-4-yl)ethylamino]cholestane (named DX115)

The first step is a synthesis of the compound 3β-octanoxycholestane comprising the following steps:

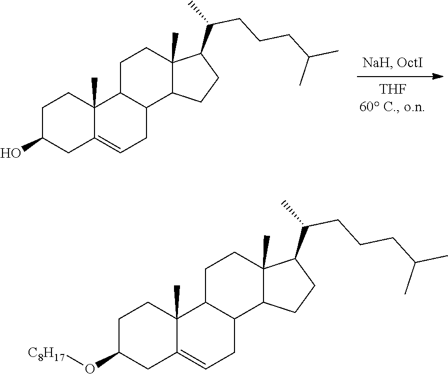

4.00 g of cholesterol were dissolved in 20 ml of tetrahydrofuran. 0.84 g of NaH was added and allowed to react for 30 minutes at 60° C., then 3.0 g of isooctane were added. The mixture thus obtained was left at 60° C. overnight. After cooling the solution, the reaction was neutralized by adding 20 ml of water. The mixture was filtered and the THF evaporated off under vacuum. The mixture was transferred into a separating funnel and the aqueous phase was extracted three times with ethyl acetate. The organic phases thus obtained were combined and dried over MgSO₄ and then evaporated to give an oil.

The oil obtained was dissolved in 2 ml of Et₂O and MeOH was added until a white precipitate was formed. The powder was filtered off, washed with cold MeOH and dried. A white powder of 2.5 g (corresponding to 48%) of 3β-octanoxycholestane was thus obtained.

¹H-NMR (500 MHz, CDCl₃): δ (ppm) 5.35 (s, 1H), 3.45-3.43 (q, 2H), 3.15-3.10 (q, 1H), 2.37-2.35 (d, 1H), 2.21-2.16 (t, 1H), 2.02-1.95 (m, 2H), 1.90-1.84 (m, 3H), 1.58-0.97 (m, 39H), 0.92-0.91 (d, 3H), 0.87-0.86 (dd, 6H), 0.67 (s, 3H).

The second step consists in synthesizing, starting with 3β-octanoxycholestane, the compound 3β-octanoxy-5,6α-epoxycholestane as follows:

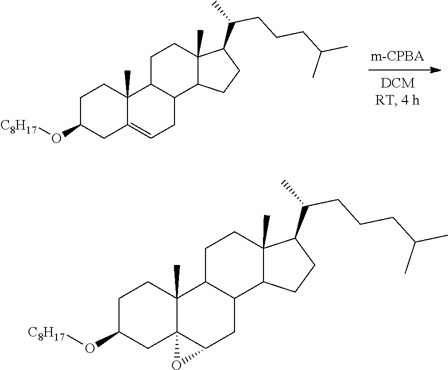

0.90 g of meta-chloroperoxybenzoic acid (corresponding to 4.0 mmol) was dissolved in 40 ml of dichloromethane and added dropwise to a mixture of 1.50 g (3.0 mmol) of 3β-octanoxycholestane dissolved in 10 ml of dichloromethane. The mixture thus obtained was stirred and maintained at room temperature for 3 hours. The mixture thus obtained was washed with an aqueous solution containing 10% by weight of Na₂S₂O₃, saturated NaHCO₃ solution and saturated NaCl solution. The organic phase was dried over anhydrous MgSO₄. Vacuum evaporation of the organic solvent was performed, to give a transparent viscous oil. 5 ml of Et₂O were added to dissolve the oil, 25 ml of MeOH were then added and the mixture was heated to the boiling point three times and finally maintained at 0° C. overnight to promote precipitation. A white powder was filtered off, washed with cold MeOH and dried: 1.19 g, corresponding to a yield of 77% (with an enantiomeric excess 90%), of 3β-octanoxy-5,6α-epoxycholestane were thus obtained.

¹H-NMR (500 MHz, CDCl₃): δ (ppm) 3.51-3.37 (m, 3H), 2.88-2.87 (d, 1H), 2.02-1.87 (m, 4H), 1.84-1.76 (m, 1H), 1.69-1.67 (m, 1H), 1.58-1.45 (m, 7H), 0.89-0.88 (m, 42H), 0.60 (s, 3H).

The third step consists in synthesizing 3β-octanoxy-5α-hydroxy-6β-[2-(1H-imidazol-4-yl)ethylamino]cholestane (DX115 in basic form) as follows:

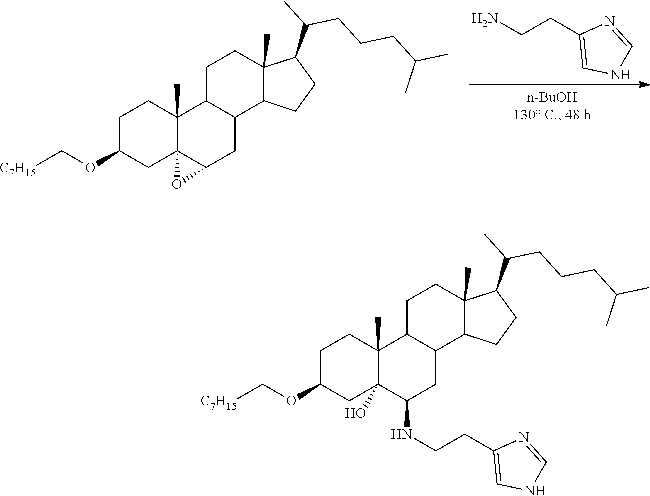

0.48 g of histamine in its basic form (corresponding to 4.31 mmol) was added to a 10 ml butanolic solution comprising 1.1 g (2.14 mmol) of the compound 3β-octanoxy-5,6α-epoxycholestane with stirring. The mixture was kept stirring at reflux, heating at a temperature of 130° C. for 48 hours.

The reaction progress can be monitored by thin layer chromatography (TLC) to follow the conversion of the 3β-octanoxy-5,6α-epoxycholestane.

After cooling, the mixture was diluted in 10 ml of methyl tert-butyl ether. The organic phase was washed twice with 10 ml of water and then once with 10 ml of saturated NaCl solution

The organic phase was dried over anhydrous MgSO₄. The mixture was purified by column chromatography on a purification machine. The eluent used was a 95/5 ethyl acetate/methanol mixture. A white powder of 0.74 g of 3β-octanoxy-5α-hydroxy-6β-[2-(1H-imidazol-4-yl)ethylamino]cholestane was obtained. The final reaction yield was 55% with a purity of greater than 95% measured by NMR (nuclear magnetic resonance) and TLC (thin layer chromatography) analysis.

¹H-NMR (500 MHz, MeOD-4d): δ (ppm) 7.59 (s, 1H), 6.86 (s, 1H), 3.79-3.74 (q, 1H), 3.49-3.47 (q, 2H), 2.95-2.90 (m, 1H), 2.78-2.70 (m, 3H), 2.40 (s, 1H), 2.01-1.84 (m, 5H), 1.62-1.54 (m, 9H), 1.39-1.02 (m, 30H), 0.95-0.89 (d, 12H), 0.70 (s, 3H).

### Example 13: Preparation of a Dilactate Salt of the Compound 3β-octanoxy-5α-hydroxy-6β-[2-(1H-imidazol-4-yl)ethylamino]cholestane (DX115 in dilactate form)

A dilactate salt of the compound 3β-octanoxy-5α-hydroxy-6β-[2-(1H-imidazol-4-yl)ethylamino]cholestane was prepared in the following manner:

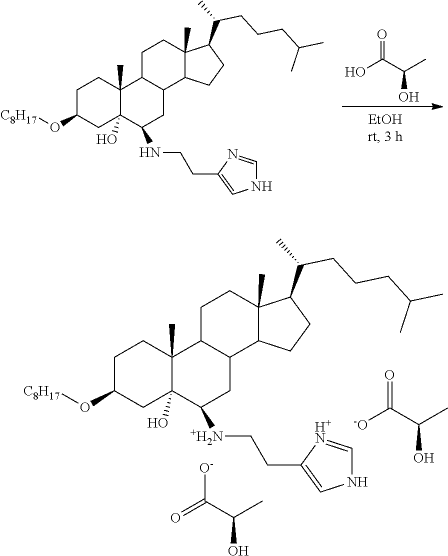

166.2 mg of lactic acid (1.85 mmol) were added to a solution of 0.57 g of 3β-octanoxy-5α-hydroxy-6β-[2-(1H-imidazol-4-yl)ethylamino]cholestane (0.92 mmol) in 5 ml of anhydrous ethanol with stirring. Stirring was continued at room temperature for 3 hours. Vacuum evaporation of the organic solvent gave a white powder of 0.59 g of 3β-octanoxy-5α-hydroxy-6β-[2-(1H-imidazol-4-yl)ethylamino]cholestane dilactate.

¹H-NMR (500 MHz, MeOD-4d): δ (ppm) 7.71 (s, 1H), 6.94 (s, 1H), 4.02-3.98 (q, 2H), 3.72-3.65 (q, 1H), 3.41-3.31 (m, 3H), 3.21-3.16 (m, 1H), 2.95-2.92 (t, 2H), 2.86-2.85 (d, 1H), 2.04-1.99 (t, 1H), 1.96-1.93 (d, 1H), 1.83-1.59 (m, 7H), 1.49-1.04 (m, 38H), 0.98-0.89 (m, 2H), 0.85-0.84 (d, 3H), 0.81-0.77 (m, 9H), 0.66 (s, 3H).

### Example 14: Synthesis of the Compound 3β-azido-5α-hydroxy-6β-[2-(1H-imidazol-4-yl)ethylamino]cholestane (named DX123)

The first step is the synthesis of the compound 3-mesylcholestane, comprising the following steps:

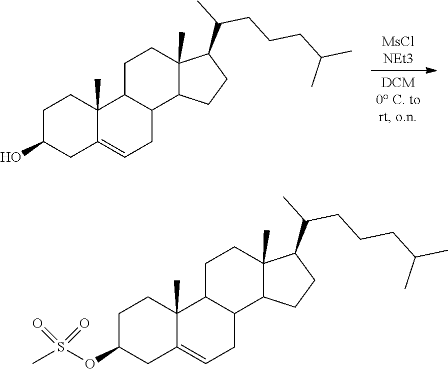

40 g of cholesterol (0.1 mol) and 22 ml of Et₃N (d=0.88 g/ml, 0.19 mol) were dissolved in 340 ml of anhydrous dichloromethane at 0° C. in a 1 L flask. 10 ml of methanesulfonyl chloride (1.48 g/ml, 0.13 mol) were dissolved in 40 ml of anhydrous dichloromethane and added dropwise to the solution containing the cholesterol. The mixture thus obtained was left under magnetic stirring overnight and allowed to warm up to room temperature.

After this time, the reaction was monitored by TLC and concentrated under vacuum to two thirds of the initial volume. Addition of 500 ml of MeOH allowed the production of 46.4 g of a white precipitate corresponding to the desired product (97% yield).

¹H-NMR (500 MHz, CDCl₃): δ (ppm) 5.42-5.41 (d, 1H), 4.55-4.49 (q, 1H), 3.00 (s, 3H), 2.56-2.45 (m, 2H), 2.05-1.96 (m, 3H), 1.92-1.75 (m, 3H), 1.60-0.93 (m, 23H), 0.92-0.90 (d, 3H), 0.87-0.85 (dd, 6H), 0.67 (s, 3H).

The second step consists in synthesizing, starting with 3β-mesylcholesterol, the compound 3β-azidocholestane as follows:

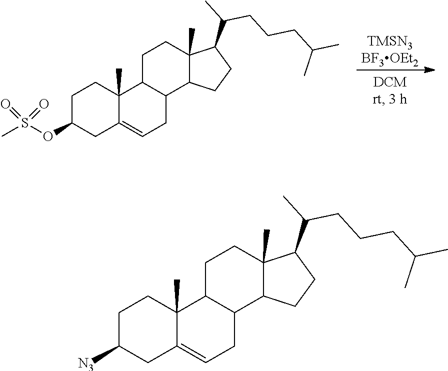 [SMILES: CC(C)CCCC(C)[C@H]1CCC2C3CC=C4C[C@@H](OS(C)(=O)=O)CC[C@]4(C)C3CC[C@@]21C ; CC(C)CCCC(C)[C@H]1CCC2C3CC=C4C[C@@H](N=[N+]=[N-])CC[C@]4(C)C3CC[C@@]21C]

The following were added in sequence to a 500 ml flask at room temperature: 23.27 g of 3β-mesylcholesterol (50.1 mmol), 100 ml of anhydrous dichloromethane, 7.5 ml of trimethylsilyl azide (d=0.868 g/ml, 56.5 mmol) and finally 12.5 ml of boron trifluoride diethyl etherate (d=1.15 g/ml, 101.3 mmol). The mixture thus obtained was stirred magnetically for 3 hours.

After this period, the reaction mixture was neutralized by adding 100 ml of 2M NaOH solution. The organic products were extracted twice with dichloromethane. The organic phases were combined and rinsed twice with saturated NaCl solution. The organic phase was dried over MgSO₄, filtered and then evaporated to give a solid. The crude reaction product was purified by column chromatography, eluting with 100% hexane. 13.33 g of a yellowish-white powder corresponding to 3β-azidocholestane were thus obtained. The final reaction yield is 65%.

¹H-NMR (500 MHz, CDCl₃): δ (ppm) 5.39-5.38 (d, 1H), 3.23-3.17 (q, 1H), 2.30-2.28 (d, 2H), 2.03-1.97 (m, 2H), 1.91-1.81 (m, 3H), 1.60-0.94 (m, 24H), 0.92-0.91 (d, 3H), 0.87-0.86 (dd, 6H), 0.68 (s, 3H).

The third synthetic step consists in synthesizing, starting with 3β-azidocholestane, the compound 3β-azido-5,6α-epoxycholestane as follows:

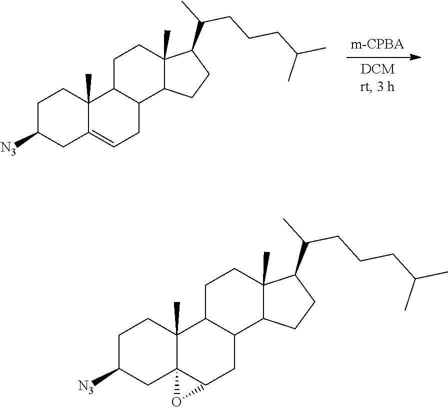

950 mg of meta-chloroperoxybenzoic acid at 77% purity (4.24 mmol) were dissolved in 15 ml of dichloromethane and added dropwise to a solution of 1.3 g of 3β-azidocholestane (3.16 mmol) dissolved in 15 ml of dichloromethane. The mixture thus obtained was stirred and maintained at room temperature for 3 hours. The mixture thus obtained was washed twice with an aqueous Na₂S₂O₃ solution at 10% by weight, twice with saturated NaHCO₃ solution and once with saturated NaCl solution. The organic phase was dried over anhydrous MgSO₄. Vacuum evaporation of the organic solvent was performed to give 1.35 g of a white powder corresponding to the mixture of: 3-azido-5,6α-epoxycholestane (83% of the total) and 3β-azido-5,6p-epoxycholestane (17% of the white powder). The final product was used without further purification.

¹H-NMR (500 MHz, CDCl₃): δ (ppm) 3.63-3.56 (q, 1H), 2.94-2.93 (d, 1H), 2.13-2.08 (t, 1H), 1.97-0.94 (m, 30H), 0.89-0.88 (d, 3H), 0.86-0.85 (dd, 6H), 0.61 (s, 3H).

The fourth step is the synthesis of 3β-azido-5α-hydroxy-6β-[2-(1H-imidazol-4-yl)ethylamino]cholestane (DX123 in neutral form) as follows:

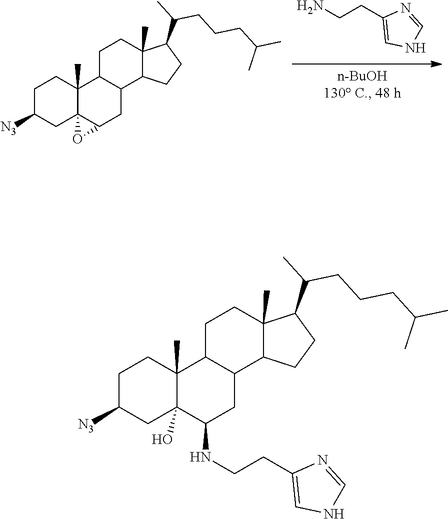

864 mg of histamine in its basic form (7.77 mmol) were added to a 20 ml butanolic solution comprising 2.02 g of the 83% compound 3β-azido-5,6α-epoxycholestane (3.9 mmol) with stirring at 130° C. The mixture was kept with stirring at reflux, heating at a temperature of 130° C. for 48 hours.

The reaction progress can be monitored by thin layer chromatography (TLC) to follow the conversion of the 3β-azido-5,6α-epoxycholestane.

After cooling, the mixture was diluted in 15 ml of methyl tert-butyl ether. The organic phase was washed three times with 15 ml of water.

The organic phase was dried over anhydrous MgSO₄, filtered and then evaporated to obtain a brown oil. The mixture was purified by column chromatography on silica gel on a purification machine including a 40 g pre-packed column, eluting with dichloromethane/ethyl acetate from 75/25% to 0/100%. A white powder of 890 mg of 3β-azido-5α-hydroxy-6β-[2-(1H-imidazol-4-yl)ethylamino]cholestane was obtained. The final reaction yield was 42% with a purity of greater than 97% measured by NMR (nuclear magnetic resonance) and TLC (thin layer chromatography) analysis.

¹H-NMR (500 MHz, MeOD-4d): δ (ppm) 7.55 (s, 1H), 6.81 (s, 1H), 3.73-3.67 (q, 1H), 2.90-2.85 (m, 1H), 2.72-2.62 (m, 3H), 2.33 (s, 1H), 2.05-2.00 (t, 1H), 1.96-1.94 (m, 1H), 1.84-1.77 (m, 1H), 1.74-1.72 (m, 1H), 1.62-0.97 (m, 27H), 0.89-0.88 (d, 3H), 0.85-0.84 (d, 6H), 0.64 (s, 3H).

### Example 15: Preparation of a dilactate salt of the compound 3β-azido-5α-hydroxy-6β-[2-(1H-imidazol-4-yl)ethylamino]cholestane (DX123 dilactate Form)

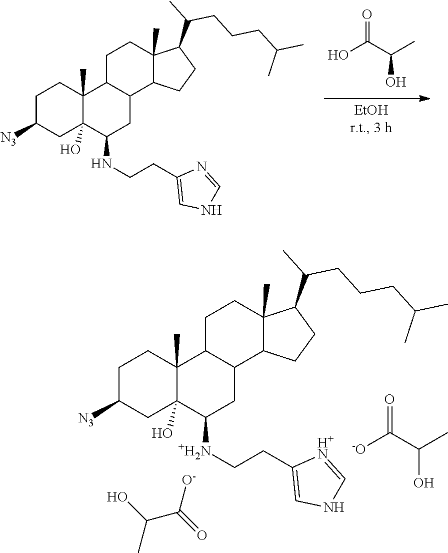

63.5 mg of lactic acid (0.77 mmol) were added to a solution of 210 mg of 3β-azido-5α-hydroxy-6β-[2-(1H-imidazol-4-yl)ethylamino]cholestane in 4 ml of anhydrous ethanol with stirring. Stirring was continued at room temperature for 3 hours. Vacuum evaporation of the organic solvent gave a white powder of 263.5 mg of 3β-azido-5α-hydroxy-6β-[2-(1H-imidazol-4-yl)ethylamino]cholestane dilactate.

¹H-NMR (500 MHz, MeOD-4d): δ (ppm) 7.67 (s, 1H), 6.92 (s, 1H), 4.01-3.97 (m, 2H), 3.73-3.67 (q, 1H), 3.34-3.29 (m, 1H), 3.19-3.13 (m, 1H), 2.91-2.88 (t, 2H), 2.81 (s, 1H), 2.20-2.15 (t, 1H), 1.94-1.92 (d, 1H), 1.77-1.75 (m, 3H), 1.66-1.58 (m, 4H), 1.47-0.98 (m, 26H), 0.95-0.87 (m, 2H), 0.83-0.82 (d, 3H), 0.77-0.76 (dd, 6H), 0.65 (s, 3H).

### Example 16: Synthesis of a trichloride salt of the compound 3β-amino-5α-hydroxy-6β-[2-(1H-imidazol-4-yl)ethylamino]cholestane (DX125 in Trichloride Form)

The reaction for synthesizing a trichloride salt of 3β-amino-5α-hydroxy-6β-[2-(1H-imidazol-4-yl)ethylamino]cholestane from 3β-azido-5α-hydroxy-6β-[2-(1H-imidazol-4-yl)ethylamino]cholestane is as follows:

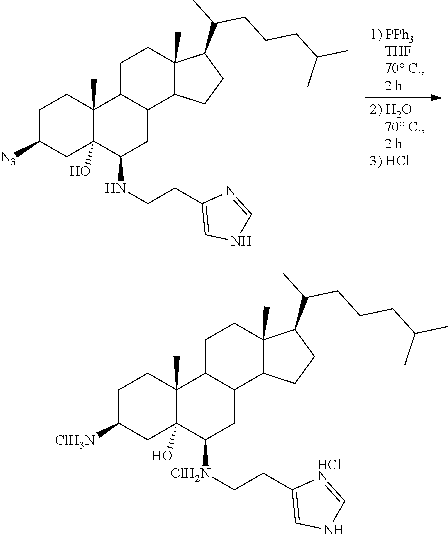

730 mg of triphenylphosphine (2.8 mmol) were added to 8.0 ml of a THF solution of 300 mg of 3β-azido-5α-hydroxy-6β-[2-(1H-imidazol-4-yl)ethylamino]cholestane (0.56 mmol) with stirring at 70° C. The mixture was kept with stirring at reflux, heating at a temperature of 70° C. for 2 hours. 0.5 ml of water (corresponding to 27.8 mmol) was then added and stirring was continued for a further two hours at 70° C. The reaction progress was monitored by thin layer chromatography (TLC), and the solvent mixture was then evaporated off. The white powder obtained was dissolved with 20 ml of dichloromethane and transferred into a separating funnel containing 20 ml of aqueous HCl solution (1 ml of 37% HCl in 19 ml of water), and the aqueous phase was washed three times with dichloromethane. The aqueous phase was dried under vacuum to obtain a white powder. The powder was taken up in dichloromethane and filtered a final time to remove the last traces of triphenylphosphine. The procedure afforded 350 mg of a trichloride salt of 3β-amino-5α-hydroxy-6β-[2-(1H-imidazol-4-yl)ethylamino]cholestane in quantitative yield and a purity of greater than 95%.

¹H-NMR (500 MHz, MeOD-4d): δ (ppm) 8.90 (s, 1H), 7.56 (s, 1H), 3.67-3.60 (q, 1H), 3.56-3.41 (m, 4H), 3.28-3.27 (d, 1H), 2.61-2.56 (t, 1H), 2.08-2.05 (d, 1H), 1.99-1.11 (m, 28H), 1.06-1.00 (dd, 1H) 0.96-0.94 (d, 3H), 0.89-0.88 (dd, 6H), 0.78 (s, 3H).

### Example 17: Synthesis of the Compound 3β-acetamido-5α-hydroxy-6β-[2-(1H-imidazol-4-yl)ethylamino]cholestane (named DX127)

The first synthetic step is the reduction of the azide group to amine in the 3-position of the cholestane 3β-azide derivative.

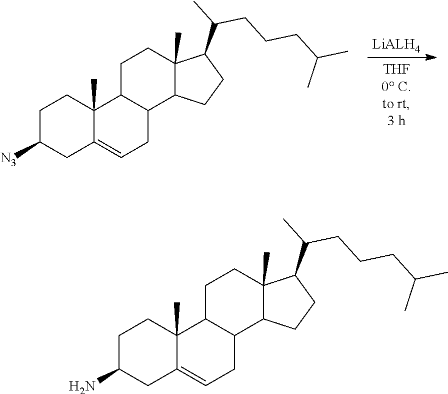 [SMILES: CC(C)CCCC(C)[C@H]1CCC2C3CC=C4C[C@@H](N=[N+]=[N-])CC[C@]4(C)C3CC[C@@]21C ; CC(C)CCCC(C)[C@H]1CCC2C3CC=C4C[C@@H](N)CC[C@]4(C)C3CC[C@@]21C]

5.21 g of cholestane 3β-azide (12.7 mmol) were dissolved in 60 ml of tetrahydrofuran (THF) and five portions of about 480 mg of LiAIH₄ were then added every 15 min for a total of 2.32 g (61.1 mmol). The mixture thus obtained was stirred magnetically for 3 hours. After this period, the reaction was neutralized by adding a few drops of aqueous 5% Na₂CO₃ (added gently). The organic phase was extracted three times with EtOAc and the organic phases were combined. The resulting solution was dried over MgSO₄, filtered and then evaporated to give a solid. A sufficiently clean white powder of 3.78 g corresponding to 3β-aminocholestane was thus obtained. The final reaction yield is 77%.

¹H-NMR (500 MHz, CDCl₃): δ (ppm) 5.32-5.31 (d, 1H), 2.63-2.57 (q, 1H), 2.17-2.13 (m, 1H), 2.08-1.93 (m, 4H), 1.85-1.81 (m, 2H), 1.72-1.68 (m, 1H), 1.43-0.84 (m, 33H), 0.68 (s, 3H).

The second step consists in synthesizing, starting with 3-aminocholestane, the compound cholestane 3β-acetamide as follows:

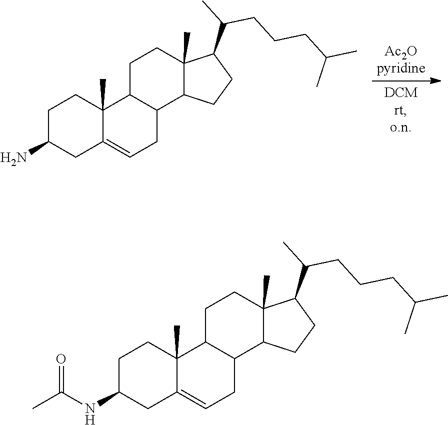

3.78 g of 3β-aminocholestane (9.8 mmol) were dissolved in 20 ml of anhydrous dichloromethane, and 16 ml of anhydrous pyridine (198 mmol) and 5.0 g of acetic anhydride (49.0 mmol) were then added to the reaction mixture. The mixture thus obtained was stirred and maintained at room temperature overnight. The mixture thus obtained was washed three times with aqueous 0.1 M HCl solution and the organic phase was dried over anhydrous MgSO₄, filtered and dried under vacuum. The oil obtained was dissolved with 30 ml of chloroform, 90 ml of MeOH were added and the mixture was heated to the boiling point three times and until the volume of solvents was reduced by two thirds, and finally maintained at 0° C. to promote precipitation. A white powder was obtained, which was filtered off, washed with cold MeOH and dried. 2.41 g, corresponding to a 58% yield of cholestane 3β-acetamide, were thus obtained.

¹H-NMR (500 MHz, CDCl₃): δ (ppm) 5.36-5.35 (d, 1H), 5.32-5.30 (d, 1H), 3.73-3.65 (q, 1H), 2.32-2.29 (d, 1H), 2.09-1.79 (m, 9H), 1.60-0.95 (m, 22H), 0.92-0.90 (d, 3H), 0.87-0.85 (dd, 6H), 0.67 (s, 3H).

The third step consists in synthesizing 5,6-epoxycholestane 3β-acetamide as follows:

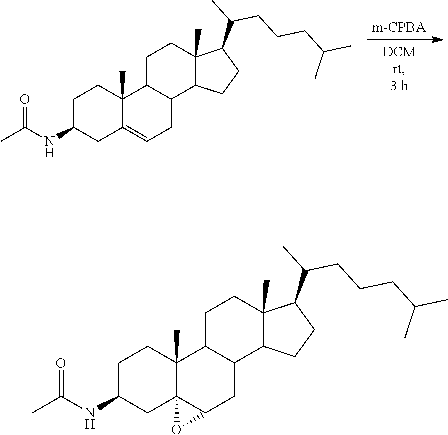

1.19 g of meta-chloroperoxybenzoic acid at 77% purity (5.3 mmol) were dissolved in 10 ml of dichloromethane and added dropwise to a mixture of 1.61 g of cholestane 3β-acetamide (3.8 mmol) dissolved in 25 ml of dichloromethane. The mixture thus obtained was stirred and maintained at room temperature for 3 hours. The mixture obtained was washed twice with an aqueous solution containing 10% by weight of Na₂S₂O₃, and twice with saturated NaHCO₃ solution and with saturated NaCl solution. The organic phase was dried over anhydrous MgSO₄. Vacuum evaporation of the organic solvent was performed to give 1.65 g of a white powder comprising: 5,6α-epoxycholestane 3β-acetamide (60% of the white powder) and 5,6p-epoxycholestane 3β-acetamide (40% of the white powder). The 5,6α-epoxycholestane 3β-acetamide was used without further purification.

¹H-NMR (500 MHz, CDCl₃): δ (ppm) 5.29-5.28 (d, 1H), 4.05-3.99 (q, 1H), 2.89-2.88 (d, 1H), 2.08-0.84 (m, 43H), 0.60 (s, 3H).

The fourth step consists in synthesizing 5α-hydroxy-6β-[2-(1H-imidazol-4-yl)ethylamino]cholestane 3β-acetamide (DX127 in neutral form) as follows:

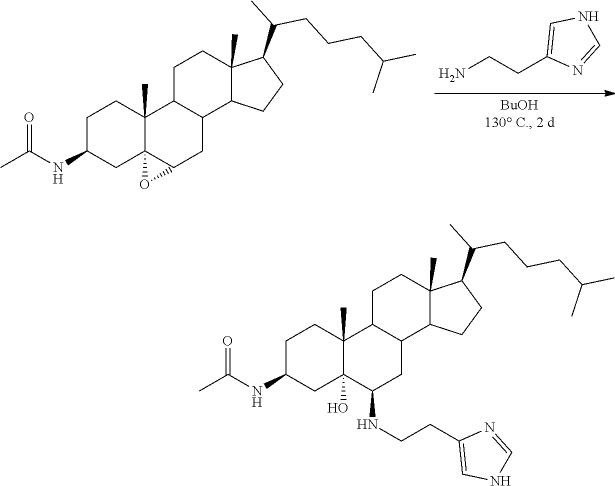

0.47 g of histamine in its basic form (corresponding to 4.26 mmol) was added to a 20 ml butanolic solution comprising 1.65 g of the compound 5,6α-epoxycholestane 3β-acetamide at 60%, corresponding to 0.99 mmol, with stirring. The mixture was kept stirring at reflux, heating at a temperature of 130° C. for 48 hours. The reaction progress can be monitored by thin layer chromatography (TLC) to follow the conversion of the 5,6α-epoxycholestane 3β-acetamide. After cooling, the mixture was diluted in 20 ml of methyl tert-butyl ether. The organic phase was washed twice with 20 ml of water and three times with 20 ml of saturated NaCl solution. The organic phase was dried over anhydrous MgSO₄. The mixture was purified by column chromatography on a purification machine. The eluent used was a 75/20/5% mixture of dichloromethane/methanol/ammonia. A white powder of 0.37 g of 5α-hydroxy-6β-[2-(1H-imidazol-4-yl)ethylamino]cholestane 3β-acetamide was obtained. The final reaction yield was 30% with a purity of greater than 97% measured by NMR (nuclear magnetic resonance) and TLC (thin layer chromatography) analysis.

¹H-NMR (500 MHz, MeOD-4d): δ (ppm) 7.56 (s, 1H), 6.81 (s, 1H), 4.15-4.08 (q, 1H), 2.91-2.88 (m, 1H), 2.74-2.68 (m, 3H), 2.35 (s, 1H), 1.99-1.94 (m, 2H), 1.87-1.77 (m, 5H), 1.66-0.97 (m, 28H), 0.90-0.88 (d, 3H), 0.85-0.84 (d, 6H), 0.65 (s, 3H).

### Example 18: Preparation of a dilactate salt of the compound 3β-acetamido-5α-hydroxy-6β-[2-(1H-imidazol-4-yl)ethylamino]cholestane (DX127 in dilactate form)

A dilactate salt of the compound 3β-acetamido-5α-hydroxy-6β-[2-(1H-imidazol-4-yl)ethylamino]cholestane was prepared in the following manner:

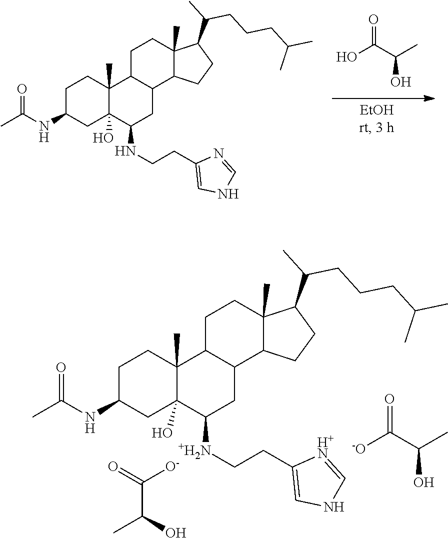

120.6 mg of lactic acid (1.34 mmol) were added to a solution of 370 mg of 5α-hydroxy-6β-[2-(1H-imidazol-4-yl)ethylamino]cholestane 3β-acetamide in 5 ml of anhydrous ethanol with stirring. Stirring was continued at room temperature for 3 hours. Vacuum evaporation of the organic solvent gave a white powder of 490 mg of 5α-hydroxy-6β-[2-(1H-imidazol-4-yl)ethylamino]cholestane 3β-acetamide dilactate.

¹H-NMR (500 MHz, MeOD-4d): δ (ppm) 7.69 (s, 1H), 6.91 (s, 1H), 4.08-4.03 (m, 1H), 3.37-3.27 (m, 1H), 3.18-3.12 (m, 2H), 2.91-2.88 (t, 2H), 2.77 (s, 1H), 2.07-2.02 (t, 1H), 1.93-1.90 (d, 1H), 1.79 (s, 3H), 1.76-0.88 (m, 36H), 0.82-0.81 (d, 3H), 0.75-074 (dd, 6H), 0.63 (s, 3H).

### Example 19: Synthesis of the Compound 3β-methylsulfonyl-5α-hydroxy-6β-[2-(1H-imidazol-4-yl)ethylamino]cholestane (named DX129)

The first step consists in synthesizing, starting with 3β-mesylcholesterol, the compound 3β-methylthiocholestane as follows:

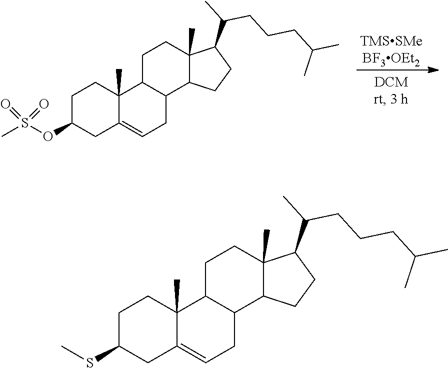

The following were added in sequence to a 500 ml flask at room temperature: 10.62 g of 3-mesylcholesterol (22.9 mmol), 50 ml of dichloromethane, 5.0 g of trimethyl(methylthio)silane (41.6 mmol), and 8.0 ml of boron trifluoride diethyl etherate (d=1.15 g/ml, 64.8 mmol). The mixture thus obtained was stirred magnetically for 3 hours.

After this period, the reaction mixture was neutralized by adding 100 ml of 2M NaOH solution. The organic phase was extracted twice with dichloromethane. The organic phases were combined and rinsed twice with saturated NaCl solution. The organic phase was dried over MgSO₄, filtered and then evaporated to obtain a solid. The crude reaction product was purified by column chromatography on silica gel, eluting with 100% hexane. A white powder of 6.04 g corresponding to 3β-methylthiocholestane was thus obtained. The final reaction yield is 63%.

¹H-NMR (500 MHz, CDCl₃): δ (ppm) 5.33 (s, 1H), 2.71-2.65 (m, 1H), 2.30-2.26 (m, 2H), 2.11 (s, 3H), 2.02-0.94 (m, 29H), 0.92-0.91 (d, 3H), 0.87-0.85 (dd, 6H), 0.67 (s, 3H).

The second synthetic step consists in synthesizing, starting with 3β-methylthiocholestane, the compound 3β-methylsulfonyl-5,6-epoxycholestane as follows:

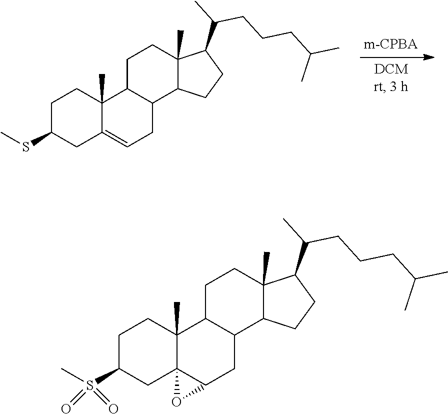

6.30 g of meta-chloroperoxybenzoic acid at 77% purity (28.1 mmol) were dissolved in 40 ml of dichloromethane and a solution of 2.9 g of 3-methylthiocholestane (6.8 mmol) in 20 ml of dichloromethane was added dropwise. The mixture thus obtained was stirred and maintained at room temperature for 3 hours. The mixture thus obtained was washed twice with aqueous Na₂S₂O₃ solution at 10% by weight, three times with saturated NaHCO₃ solution and once with saturated NaCl solution. The organic phase was dried over anhydrous MgSO₄. Vacuum evaporation of the organic solvent was performed to obtain 2.10 g of a white powder. The crude reaction product was purified by column chromatography, eluting initially with 100% hexane and then with mixtures of hexane and EtOAc. The desired product was purified by column chromatography on silica gel, eluting with 55%/45% hexanes/EtOAc. A white powder of 380 mg corresponding to 3-methylthio-5,6-epoxycholestane was thus obtained. The final reaction yield is 12%.

¹H-NMR (500 MHz, CDCl₃): δ (ppm) 3.25-3.20 (m, 1H), 3.02-3.01 (d, 1H), 2.83 (s, 3H), 2.11-2.09 (m, 1H), 1.98-1.93 (m, 3H), 1.87-1.79 (m, 3H), 1.57-0.93 (m, 24H), 0.89-0.88 (d, 3H), 0.86-0.85 (dd, 6H), 0.61 (s, 3H).

The third step is the synthesis of 3β-methylsulfonyl-5α-hydroxy-6β-[2-(1H-imidazol-4-yl)ethylamino]cholestane (DX129 in basic form) as follows:

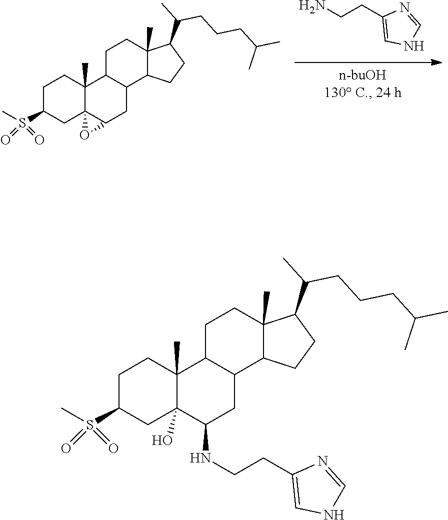

338 mg of histamine in its basic form (3.04 mmol) were added to a 5 ml butanolic solution comprising 350 mg of the compound 3-methylsulfonyl-5,6-epoxycholestane (0.75 mmol) with stirring at 130° C. The mixture was kept stirring at reflux, heating at a temperature of 130° C. for 48 hours. The reaction progress can be monitored by thin layer chromatography (TLC) to follow the conversion of the 3-methylsulfonyl-5,6-epoxycholestane.

After cooling, the mixture was diluted in 5 ml of methyl tert-butyl ether. The organic phase was washed three times with 15 ml of saturated sodium chloride.

The organic phase was dried over anhydrous MgSO₄, filtered and then evaporated to give a brown oil. The crude reaction product was purified by column chromatography, eluting initially with 100% EtOAc, and then with EtOAc/MeOH mixtures. The desired product was purified with a 75%/25% EtOAc/MeOH mixture. A yellow powder of 190 mg corresponding to 3-methylsulfonyl-5α-hydroxy-6β-[2-(1H-imidazol-4-yl)ethylamino]cholestane was obtained. The product was purified a second time by column chromatography to obtain a purity of greater than 97% measured by NMR (nuclear magnetic resonance) and TLC (thin layer chromatography) analysis.

167.4 mg of a white powder were obtained. The final reaction yield is 39%.

¹H-NMR (500 MHz, MeOD-4d): δ (ppm) 7.63 (s, 1H), 6.89 (s, 1H), 3.49-3.40 (m, 1H), 3.04-3.02 (m, 1H), 2.89 (s, 3H), 2.81-2.78 (m, 3H), 2.50 (s, 1H), 2.44-2.40 (t, 1H), 2.02-2.01 (m, 1H), 1.94-1.92 (m, 1H), 1.88-1.83 (m, 1H), 1.76-1.00 (m, 30H), 0.90-0.89 (d, 3H), 0.85-0.84 (d, 6H), 0.66 (s, 3H).

### Example 20: Preparation of a dilactate salt of the compound 3β-methylsulfonyl-5α-hydroxy-6β-[2-(1H-imidazol-4-yl)ethylamino]cholestane (DX129 in Dilactate Form)

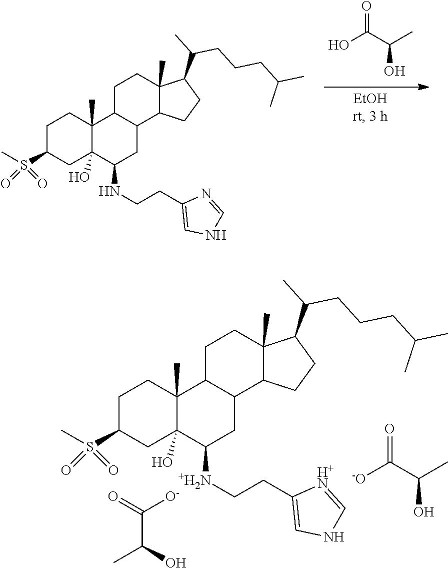

51.6 mg of lactic acid (1.34 mmol) were added to a solution of 165.0 mg of 3β-methylsulfonyl-5α-hydroxy-6β-[2-(1H-imidazol-4-yl)ethylamino]cholestane in 5 ml of anhydrous ethanol with stirring. Stirring was continued at room temperature for 3 hours. Vacuum evaporation of the organic solvent gave a white powder of 216.6 mg of 3β-methylsulfonyl-5α-hydroxy-6β-[2-(1H-imidazol-4-yl)ethylamino]cholestane dilactate.

¹H-NMR (500 MHz, MeOD-4d): δ (ppm) 7.77 (s, 1H), 6.98 (s, 1H), 3.52-3.47 (m, 1H), 3.45-3.41 (m, 1H), 3.18-3.14 (m, 1H), 2.96-2.94 (t, 2H), 2.89-2.87 (m, 4H), 2.59-2.55 (t, 1H), 2.02-2.00 (m, 1H), 1.97-1.95 (m, 1H), 1.86-1.80 (m, 2H), 1.75-1.65 (m, 5H) 1.57-0.95 (m, 29H), 0.90-0.89 (d, 3H), 0.84-0.82 (dd, 6H), 0.72 (s, 3H).

### Example 21: Pharmacokinetic study of DX103

The following study is an LC/MS assay in plasma of the various molecules over 3 days (11 measurement points in the end). The graphs are always presented in comparison with DX101 which is the reference.

Protocol

| | Group 1 | Group 2 |
|---|---|---|
| Administration of | DX101 | DX103 |
| the compound | | |
| Dose | 50 mg/kg | 50 mg/kg |
| Application route | oral | oral |
| Animals | rat | rat |
| Group size | 3 | 3 |
| Samples | plasma | plasma |
| Assay | DX101 | DX103 and DX101 |

Plasma sampling at 0 (without injection), 5, 10, 15, 30 min, 1 h, 4 h, 8 h, 24 h, 48 h, 72 h (11 points)

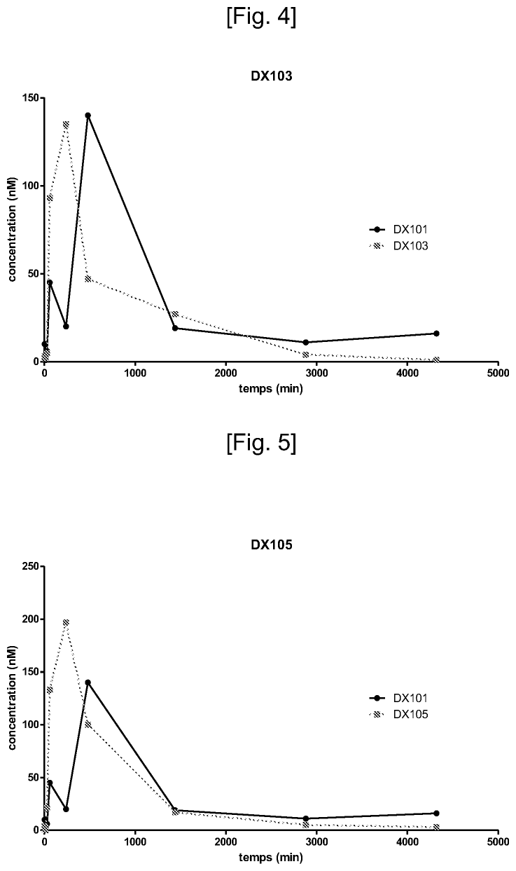

The pharmacokinetic profile of DX103 in comparison with DX101 is given in FIG. 4. The results are as follows:

| | DX101 | DX103 |
|---|---|---|
| Area under the curve | 143297 | 105351 |
| Max concentration (nM) | 140 | 135 |
| Time for Cmax (min) | 480 | 240 |

Conclusion: The profile of DX103 shows faster absorption in the body and slightly lower bioavailability than DX101.

### Example 22: Pharmacokinetic Study of DX105

The following study is an LC/MS assay in plasma of the various molecules over 3 days (11 measurement points in the end). The graphs are always presented in comparison with DX101 which is the reference.

Protocol

| | Group 1 | Group 2 |
|---|---|---|
| Administration of | DX101 | DX105 |
| the compound | | |
| Dose | 50 mg/kg | 50 mg/kg |
| Application route | oral | oral |
| Animals | rat | rat |
| Group size | 3 | 3 |
| Samples | plasma | plasma |
| Assay | DX101 | DX105 and DX101 |

Plasma sampling at 0 (without injection), 5, 10, 15, 30 min, 1 h, 4 h, 8 h, 24 h, 48 h, 72 h (11 points)

The pharmacokinetic profile of DX105 in comparison with DX101 is given in FIG. 5. The results are as follows:

| | DX101 | DX105 |
|---|---|---|
| Area under the curve | 143297 | 145590 |
| Max concentration (nM) | 140 | 197 |
| Time for Cmax (min) | 480 | 240 |

DX105 shows bioavailability that is equivalent to (or even slightly higher than) that of DX101. On the other hand, it shows much faster absorption and a much higher maximum concentration, which makes it possible to envisage good in vivo potential.

### Example 23: Pharmacokinetic Study of DX111

The following study is an LC/MS assay in plasma of the various molecules over 3 days (11 measurement points in the end). The graphs are always presented in comparison with DX101 which is the reference.

| | Group 1 | Group 2 |
|---|---|---|
| Administration of | DX101 | DX111 |
| the compound | | |
| Dose | 50 mg/kg | 50 mg/kg |
| Application route | oral | oral |
| Animals | rat | rat |
| Group size | 3 | 3 |
| Samples | plasma | plasma |
| Assay | DX101 | DX111 and DX101 |

Plasma sampling at 0 (without injection), 5, 10, 15, 30 min, 1 h, 4 h, 8 h, 24 h, 48 h, 72 h (11 points)

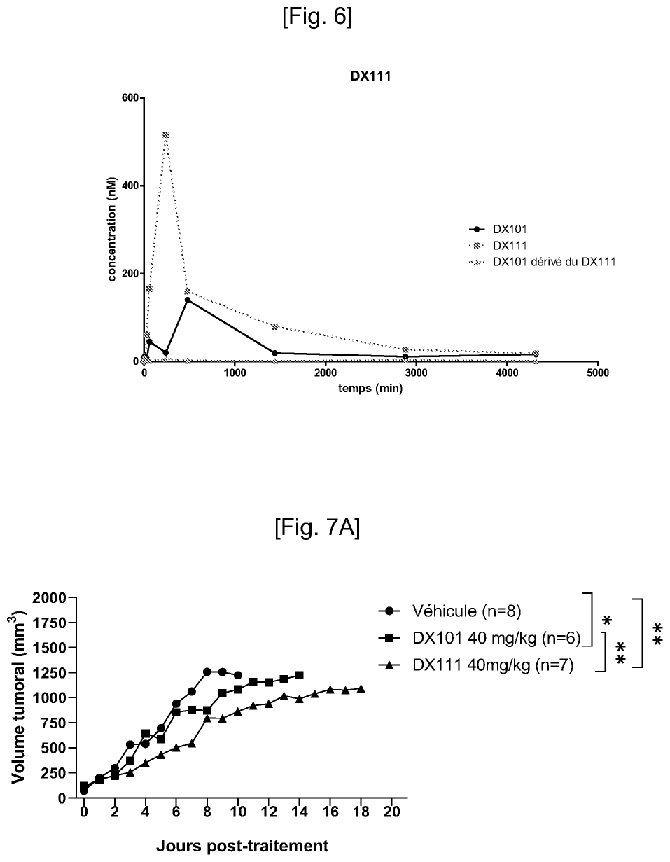

The pharmacokinetic profile of DX111 in comparison with DX101 is given in FIG. 6. The results are as follows:

| | DX101 | DX111 |
|---|---|---|
| Area under the curve | 143297 | 368921 |
| Max concentration (nM) | 140 | 515 |
| Time for Cmax (min) | 480 | 240 |

This oral pharmacokinetic study shows that DX111 has a three-fold higher absorption than DX101. In addition, DX111 has a higher maximum concentration and also faster absorption.

### Example 24: Pharmacokinetic Study of DX123

The following study is an LC/MS assay in plasma of the various molecules over 3 days (11 measurement points in the end). The graphs are always presented in comparison with DX101 which is the reference.

Protocol

| | Group 1 | Group 2 |
|---|---|---|
| Administration of | DX101 | DX123 |
| the compound | | |
| Dose | 50 mg/kg | 50 mg/kg |
| Application route | oral | oral |
| Animals | rat | rat |
| Group size | 3 | 3 |
| Samples | plasma | plasma |
| Assay | DX101 | DX123 and DX101 |

Plasma sampling at 0 (without injection), 5, 10, 15, 30 min, 1 h, 4 h, 8 h, 24 h, 48 h, 72 h (11 points)

The pharmacokinetic profile of DX123 in comparison with DX101 is given in FIG. 8. The results are as follows:

| | DX101 | DX123 |
|---|---|---|
| Area under the curve | 3019 | 6711 |
| Max concentration (nM) | 150 | 721 |
| Time for Cmax (min) | 4 | 4 |

This first oral pharmacokinetic analysis shows that DX123 has a twofold higher bioavailability compared to DX101. These results makes it possible to envisage good in vivo potential for DX123.

### Example 25: Cytotoxicity Study of DX101 Analogs According to the Invention on 4T1 Cells

Cell viability tests were performed on murine 4T1 mammary tumor cells characterized as triple negative (HER2-, ER-, PR-).

For this experiment, a cell culture medium was prepared. The culture medium consisted of Dulbecco's Modified Eagle Medium (DMEM, sold by Westburg as LO BE12-604F), comprising 4.5 g/L glucose with L-glutamine, to which 10% fetal calf serum (FCS) and 50 U/ml penicillin/streptomycin are added. The 4T1 cells were introduced into this culture medium.

96-well plates were seeded with 2000 4T1 cells per well. After 72 hours (h) of culture under normal condition, i.e. in an incubator at 37° C. at 5% O₂, the 4T1 cells were treated for 48 h with DX101, DX103, DX111, DX123, DX125, DX127 and DX129 at 100 nM, 1 μM, 2.5 μM and 10 μM. A control condition (CTL) is also performed in parallel using the previously described protocol without treatment with the molecules DX101, DX103, DX111, DX123, DX125, DX127 or DX129.

The cell viability is measured by three different methods. For the first method, MTT labeling is performed at 48 hours. This test is based on the use of the tetrazolium salt MTT (3-(4,5-dimethylthiazol-2-yl)-2,5-diphenyltetrazolium bromide). Tetrazolium is reduced by mitochondrial succinate dehydrogenase in active live cells to formazan, a purple colored precipitate. The amount of precipitate formed is proportional to the amount of live cells but also to the metabolic activity of each cell. Thus, a simple determination of the optical density at 550 nm by spectroscopy makes it possible to determine the relative amount of live and metabolically active cells. After 48 hours, the medium is aspirated, and the cells are incubated with MTT (0.5 mg/ml in culture medium) for about 3 hours. The MTT solution is aspirated and the purple crystals are dissolved in dimethyl sulfoxide (DMSO). The OD (optical density) is measured at 550 nm. The percentage of viability is then determined in each well against the CTL and the IC₅₀ (concentration at which 50% of cells remain alive) is determined for each molecule with the Prism software using a nonlinear regression curve (log(inhibitor) vs. Response).

For the second method, the percentage of viability is determined by assaying the activity of the enzyme LDH (lactate dehydrogenase) in cell supernatants using the non radioactive cytotoxicity assay kit (Promega). LDH is an enzyme released in the supernatant of dead cells. The higher the LDH activity in the supernatant, the greater the cell death. In this enzyme assay, the LDH released converts a violet tetrazolium salt to a red colored formazan, absorbing at 490 nm. The intensity of the red color is proportional to the number of dead cells. After 48 h of treatment, the supernatants are transferred to a new 96-well plate and incubated for 30 minutes in the presence of substrate mix at room temperature. The reaction is stopped with stop solution reagent and the absorbance is determined at 490 nM. The percentage of cell death is determined here using a 100% maximum LDH activity control (made from untreated cells incubated in the presence of lysis solution for 45 minutes at 37° C. just prior to addition of the substrate mix), and the cell viability in each well is then deduced from this percentage. The IC₅₀ is then determined as explained in the preceding paragraph.

For the third method, the percentage viability is determined using the CellTox Green Cytotoxicity Assay kit (Promega). This assay measures cell death via a change in membrane integrity. The assay uses a cyanine probe that does not penetrate cells when they are alive, but which binds to the DNA of dead cells, which are permeable to the probe, making the DNA fluorescent. Consequently, the higher the fluorescence in the wells, the greater the cell death. After 48 h of treatment, the cells are incubated for a minimum of 15 minutes in the presence of Celltox green reagent at room temperature and the fluorescence is read at λₑₘᵢₛₛᵢₒₙ 485 nm/λ_{excitation} 590 nm. The percentage of cell death is determined using the 100% cell death control (made from untreated cells incubated in the presence of the lysis solution for 30 minutes at 37° C. prior to the addition of Celltox green reagent), and the cell viability in each well is then deduced from this percentage. The IC₅₀ is then determined as explained previously.

The IC₅₀ results for these tests are presented in Tables 1a, 1b, and 1c. In these tables:
a The significance level was calculated by comparing the LogIC₅₀ of the compound to the LogIC₅₀ of DX101 with min n=3 and a one-way ANOVA test followed by a Dunn's post-test
n^{b} represents the number of independent tests with 4 to 10 replicates for each condition.

TABLE 1a

| | | MTT | | |
|---|---|---|---|---|
| | | Mean | | |
| IC₅₀ (μM) | | LogIC₅₀ | IC₅₀ | |
| Compound | n^{b} | (±SEM) | (μM) | p value^{a} |
| DX101 | 10 | 0.64 ± 0.05 | 4.40 | / |
| DX103 | 2 | 0.50 ± 0.02 | 3.16 | / |
| DX111 | 5 | 0.23 ± 0.03 | 1.69 | <0.05 |
| DX115 | 3 | 1.57 ± 0.12 | >10 | <0.001 |
| DX123 | 6 | 0.77 ± 0.04 | 5.94 | >0.05 |
| DX125 | 4 | 2.16 ± 0.23 | >10 | <0.0001 |
| DX127 | 2 | 1.07 ± 0.04 | 11.67 | / |
| DX129 | 1 | 1.18 ± 0.17 | 15.10 | / |

TABLE 1b

| | | LDH | | |
|---|---|---|---|---|
| | | Mean | | |
| IC₅₀ (μM) | | LogIC₅₀ | IC₅₀ | |
| Compound | n^{b} | (±SEM) | (μM) | p value^{a} |
| DX101 | 10 | 0.51 ± 0.07 | 3.24 | / |
| DX103 | 2 | 0.64 ± 0.09 | 4.40 | >0.05 |
| DX111 | 5 | 0.27 ± 0.13 | 1.88 | >0.05 |
| DX115 | 3 | 1.24 ± 0.07 | >10 | >0.05 |
| DX123 | 5 | 0.16 ± 0.19 | 1.45 | >0.05 |
| DX125 | 3 | 1.56 ± 0.63 | >10 | <0.01 |
| DX127 | 2 | 0.85 ± 0.44 | 7.09 | / |
| DX129 | 1 | 0.59 ± 0.18 | 3.87 | / |

TABLE 1c

| | | Green Cytotoxicity Assay | | |
|---|---|---|---|---|
| | | Mean | | |
| IC₅₀ (μM) | | LogIC₅₀ | IC₅₀ | |
| Compound | n^{b} | (±SEM) | (μM) | p value^{a} |
| DX101 | 8 | 0.43 0.08 | 2.68 | / |
| DX103 | 1 | 0.42 ± 0.13 | 2.63 | >0.05 |
| DX111 | 9 | 0.34 ± 0.11 | 2.18 | >0.05 |
| DX115 | 3 | 1.40 ± 0.05 | >10 | <0.001 |
| DX123 | 6 | 0.32 ± 0.13 | >10 | >0.05 |
| DX125 | 4 | 1.35 ± 0.25 | >10 | <0.001 |
| DX127 | 2 | 1.04 ± 0.13 | 10.89 | / |
| DX129 | 1 | 1.21 ± 0.10 | 16.18 | / |

It is illustrated in Tables 1a, 1 b, and 1c that for DX111, the IC₅₀ is significantly lower (up to a factor of 2.5) than that of DX101, indicating a cytotoxic activity higher than that of DX101. In addition, the activity of DX123 has a tendency to be higher than that of DX101, and the activities of DX125, DX127, and DX129 are lower than that of DX101.

### Example 26: Cytotoxicity Study of DX101 Analogs According to the Invention on BT-474 Cells

Cell viability tests were also performed on BT-474 human mammary tumor cells (characterized as triple positive HER2+, ER+, PR+). The BT-474 cells were in a cell culture medium identical to the previous example and seeded in 24-well plates at 70 000 cells per well for cell viability determination using trypan blue, or in 96-well plates at 13 000 cells per well for cell viability determination using the MTT or LDH assay. After 96 hours (h) of culture under normal conditions, i.e. in an incubator at 37° C. with 5% O₂, BT-474 cells were treated for 48 h with DX101, DX103, DX105, DX111, DX123 and DX127 at 100 nM, 1 μM, 2.5 μM and 10 μM. A control is also performed using the protocol described previously without treatment with DX101, DX103, DX105, DX111, DX123 and DX127.

After a 10-minute trypsin digestion at 37° C., the cell survival was also quantified by means of a trypan blue test with automatic counting using the Biorad TC20 machine (TC20™ Automated Cell Counter). The trypan blue test is based on the integrity of cell membranes, which is disrupted in the dead cells. Trypan blue stains dead cells blue. The Biorad TC20 cell counter counts the proportion of blue and non-blue cells, and reports the percentage of cells. The percentage of viability is then determined in each well relative to the untreated cells and the IC₅₀ is determined as explained in the preceding example. The results are represented in Table 2. Also, the percentage viability of the BT-474 cells was determined using the MTT and LDH assay, performed as described in the preceding example.

The results are represented in Tables 2a, 2b and 2c. In these tables:
a The significance level was calculated by comparing the LoglC₅₀ of the compound to the LoglC₅₀ of DX101 with min n=3 and a one-way ANOVA test followed by a Dunn's post-test (except for the LDH test where the p-value was calculated by means of a t-test)
n^{b} represents the number of independent tests with 3 to 10 replicates for each condition.

TABLE 2a

| | | MTT | | |
|---|---|---|---|---|
| | | Mean | | |
| IC₅₀ (μM) | | LogIC₅₀ | IC₅₀ | |
| Compound | n^{b} | (±SEM) | (μM) | p value^{a} |
| DX101 | 6 | 0.88 ± 0.03 | 7.53 | / |
| DX103 | 4 | 0.85 ± 0.05 | 7.08 | >0.05 |
| DX105 | 5 | 0.86 ± 0.04 | 4.08 | >0.05 |
| DX111 | 5 | 0.95 ± 0.10 | 8.94 | >0.05 |

TABLE 2b

| | | LDH | | |
|---|---|---|---|---|
| | | Mean | | |
| IC₅₀ (μM) | | LogIC₅₀ | IC₅₀ | |
| Compound | n^{b} | (±SEM) | (μM) | p value^{a} |
| DX101 | 6 | 1.03 ± 0.06 | 10.64 | / |
| DX103 | 0 | / | / | / |
| DX105 | 0 | / | / | / |
| DX111 | 3 | 0.89 ± 0.04 | 7.82 | >0.05 |

TABLE 2c

| | | Trypan Blue | | |
|---|---|---|---|---|
| | | Mean | | |
| IC₅₀ (μM) | | LogIC₅₀ | IC₅₀ | |
| Compound | n^{b} | (±SEM) | (μM) | p value^{a} |
| DX101 | 6 | 0.61 ± 0.20 | 4.03 | / |
| DX103 | 4 | 0.42 ± 0.25 | 2.62 | >0.05 |
| DX105 | 5 | 0.55 ± 0.14 | 3.57 | >0.05 |
| DX111 | 6 | 0.78 ± 0.15 | 5.96 | >0.05 |

It is illustrated in Tables 2a, 2b and 2c that the activity of the molecules DX103, DX105, DX111 and DX127 is similar to that of DX101, and that that of DX123 tends to be superior to that of DX101 in this line. All of these molecules are therefore envisaged as being good candidates for industrial development since they have biological data similar or superior to those of DX101.

### Example 27: Effect of the Analog Compound DX111 on Tumor Growth In Vivo

All the animal procedures were conducted according to our institutional guidelines after approval by the ethics committee. The 4T1 cells were cultured as previously, dissociated in trypsin, washed twice with cold PBS and resuspended in 1.5 million/ml PBS. 4T1 tumors were obtained by subcutaneous transplantation of 0.150 million cells in 100 μL into the flank of female Balb/c mice (9 weeks old, January). When the tumors reached a volume of 50-100 mm³, the mice were gavaged with 40 mg/kg of DX101 or 40 mg/kg of DX111 or the control vehicle (water). Treatment was performed daily until the end of the experiment (tumor volume >1000 mm³). Tumor volume was determined daily using a caliper and calculated using the formula: ½×(Length*Width²). The percentage of tumor growth inhibition was determined using the following formula: 100×(1-(Tumor volume, day 7/tumor volume day 0)_{DX111})/(1-(Tumor volume, day 7/tumor volume day 0)_{vehicle}).

The Kaplan-Meier method was used to compare animal survival.

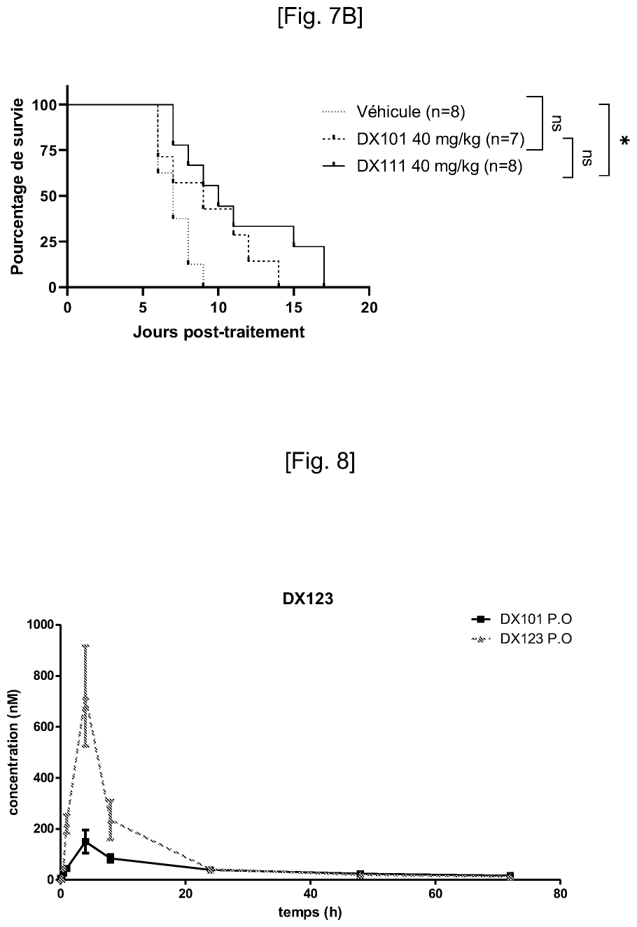

It is illustrated in FIG. 7A that DX111 shows a superior effect to DX101 on tumor growth reduction (**p<0.01, one-way ANOVA test and Tukey's post-test). Inhibition of tumor growth at 7 days was further determined to be 67% for the DX111-treated animals and 48% for the DX101-treated animals.

In addition, analysis of animal survival, also shown in FIG. 7B, indicates a better median survival of the DX111-treated animals (Log-rank Mantel-Cox test, *p<0.05 and ns, not significant; Log-rank test for trend, **p<0.01). Also, it is observed that after 15 days of treatment, survival is 25% for animals treated with DX111 while it is 0% for the animals treated with DX101. The median survival after treatment with DX101 (40 mg/kg) was 9 days while that of DX111 (40 mg/kg) was 10 days.

In conclusion, the effect of DX111 is much greater in vivo on tumor growth inhibition and strongly influences the survival of the animals.

### Example 28: Study to Determine the Pharmacokinetics and Bioavailability of DX111 Orally in Rats

Protocol: The study was performed
on four groups described below.

| | Group 1 | Group 2 | Group 3 | Group 4 |
|---|---|---|---|---|
| Administration of | DX101 | DX101 | DX111 | DX111 |
| the compound | | | | |
| Dose | 50 mg/kg | 5 mg/kg | 50 mg/kg | 5 mg/kg |
| Application route | oral | I.V. | oral | I.V. |
| Animals | rat | rat | rat | rat |
| Group size | 3 | 3 | 3 | 3 |
| Samples | plasma | plasma | plasma | plasma |
| Assay | DX101 | DX101 | DX111 | DX111 |

Plasma sampling at 0 (without injection), 15, 30 min, 1 h, 4 h, 8 h, 24 h, 48 h, 72 h

The results are given in Table 3.

TABLE 3

| Compound | DX101 | DX111 | | DX101 | DX111 |
|---|---|---|---|---|---|
| Dose po | 50 | 50 | Dose iv | 5 | 5 |
| (mg/kg) | | | (mg/kg) | | |
| Cmax [ng/ml] | 77 | 309 | Cmax [ng/ml] | 3308 | 2970 |
| Tmax [h] | 4 | 4 | | | |
| ASC(t0-tlast) | 1546 | 3228 | ASC(t0-tlast) | 6205 | 3516 |
| | | | Vd [L/kg] | 29 | 43 |
| | | | Vss [L/kg] | 19 | 12 |
| T½ 2nd phase | 39 | 24 | T½ 2nd phase | 28 | 21 |
| [h] | | | [h] | | |
| Clearance | 412 | 240 | Clearance | 11 | 25 |
| [ml/min/kg] | | | [ml/min/kg] | | |
| F % | 3% | 10% | | | |

Surprisingly, the results show that the analog compound DX111 has a three-fold higher bioavailability than the reference compound DX101 by decreasing the elimination half-life. The maximum plasma concentration obtained with DX111 is four times higher than that of DX101 and the clearance is halved.

Although the invention has been described in relation with several particular embodiments, it is quite obvious that it is not in any way limited thereto and that it encompasses all the technical equivalents of the means described and also combinations thereof if they fall within the context of the invention.

The use of the verb “contain”, “comprise” or “include” and its conjugated forms does not exclude the presence of elements or steps other than those stated in a claim.

1. A compound of formula (I): 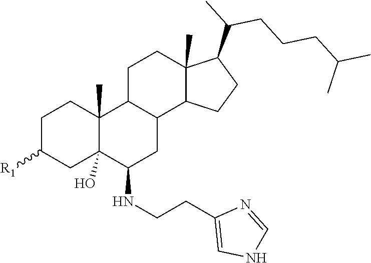
or a pharmaceutically acceptable salt of such a compound, in which:
R₁ in β position is chosen from:
F and the compound of formula (I) is 3β-fluoro-5α-hydroxy-6β-[2-(1H-imidazol-4-yl)ethylamino]cholestane,
N₃ and the compound of formula (I) is 5α-hydroxy-6β-[2-(1H-imidazol-4-yl)ethylamino]-3β-azide,
OCₙH₂ₙ₊₁,
NR₂R₃, with R₂ is H or COCₙH₂ₙ₊₁ and R₃═H,
SO₂R₂, with R₂ is H or CₙH₂ₙ₊₁
with n≤8, the compound of formula (I) being not 5α-hydroxy-6β-[2-(1H-imidazol-4-yl)ethylamino]cholestan-3β-ol,
for use as a medicament for shrinking a mammalian cancerous tumor.

2. (canceled)

3. The compound as claimed in claim 1, in which the compound of formula (I) is 5α-hydroxy-6β-[2-(1H-imidazol-4-yl)ethylamino]-3b-acetamide.

4. The compound as claimed in claim 1, in which the compound of formula (I) is 5α-hydroxy-6β-[2-(1H-imidazol-4-yl)ethylamino]-3b-amine.

5. (canceled)

6. (canceled)

7. The compound as claimed in claim 1, in which the compound of formula (I) is an O-alkyl analog and is chosen from:
3β-methoxy-5α-hydroxy-6β-[2-(1H-imidazol-4-yl)ethylamino]cholestane
3β-ethoxy-5α-hydroxy-6β-[2-(1H-imidazol-4-yl)ethylamino]cholestane
3β-octanoxy-5α-hydroxy-6β-[2-(1H-imidazol-4-yl)ethylamino]cholestane.

8. The compound as claimed in claim 1, in which the compound of formula (I) is 3b-methylsulfonyl-5α-hydroxy-6β-[2-(1H-imidazol-4-yl)ethylamino]-cholestane.

9. The compound as claimed in claim 1, in which the cancerous tumor is a chemosensitive cancer.

10. The compound as claimed in claim 1, in which the cancerous tumor is a chemoresistant cancer.

11. The compound as claimed in claim 10, in which the chemoresistant cancer is a hematological or blood cancer, lymphoma, and multiple myeloma.

12. The compound as claimed in claim 10, in which the cancer is chemoresistant to daunorubicin, cytarabine, fluorouracil, cisplatin, all-trans-retinoic acid, arsenic trioxide, bortezomib, or a combination thereof.

13. A pharmaceutical composition comprising, in a pharmaceutically acceptable vehicle, at least one compound as claimed in claim 1, for use in shrinking a mammalian cancerous tumor.

14. The pharmaceutical composition as claimed in claim 13, also comprising at least one other therapeutic agent.

15. The pharmaceutical composition as claimed in claim 14, in which the other therapeutic agent is an antineoplastic agent.

16. The pharmaceutical composition as claimed in claim 15, for use in the treatment of cancer in a patient suffering from a tumor that is chemoresistant to the antineoplastic agent when not administered in combination with the compound.

17. The pharmaceutical composition as claimed in claim 15, for use in the treatment of cancer in a patient suffering from a tumor that is chemosensitive to the antineoplastic agent, in which the dose of the antineoplastic agent administered to the patient in combination with the compound or a pharmaceutically acceptable salt thereof is less than the dose of the antineoplastic agent when not administered in combination with the compound.

18. The pharmaceutical composition as claimed in claim 13, in which the composition is in a form that is suitable for administration via any route.

19. The compound as claimed in claim 11, in which the hematological or blood cancer is leukemia.

20. The compound as claimed in claim 19, in which the leukemia is acute myeloid leukemia or acute lymphocytic leukemia.

21. The compound as claimed in claim 11, in which the lymphoma is non-Hodgkin's lymphoma.

22. The pharmaceutical composition as claimed in claim 18, in which the administration route is via an intravenous, subcutaneous, intraperitoneal or oral route.

23. A method of treating a mammalian cancerous tumor, comprising the step of administering a pharmaceutically effective amount of the pharmaceutical composition as claimed in claim 13 and shrinking the mammalian cancerous tumor.
